(19) United States
(12) Patent Application Publication
(10) Pub. No.: US 2026/0229318 A1
(43) Pub. Date: Aug. 6, 2026

(54) INFORMATION PROCESSING METHOD, PRESENTATION METHOD, PROGRAM, AND MANUFACTURING METHOD

(71) Applicant: Panasonic Intellectual Property Management Co., Ltd., Osaka (JP)

(72) Inventors: Takahiro AOKI, Osaka (JP); Hironori TSUNOYAMA, Osaka (JP); Takahiko HASHIMOTO, Shiga (JP); Akira HIWATA, Shiga (JP)

(21) Appl. No.: 19/631,162

(22) Filed: Mar. 27, 2026

Related U.S. Application Data
(63) Continuation of application No. PCT/JP2024/034654, filed on Sep. 27, 2024.

(30) Foreign Application Priority Data
Sep. 29, 2023 (JP) 2023-170464

(51) Int. Cl. G16C 20/30; G16C 10/00; G16C 20/70; G16C 20/80

(52) U.S. Cl. CPC G16C 20/30; G16C 10/00; G16C 20/70; G16C 20/80

(57) ABSTRACT

An information processing method is executed by a computer system, and includes: acquiring one or more chemical formulas of respective one or more types of molecules constituting a substance and a composition ratio of the one or more types of molecules; calculating, from the one or more chemical formulas of the respective one or more types of molecules, one or more molecular feature values of the respective one or more types of molecules; and generating physical property change data representing a change in a physical property value of the substance by using the composition ratio and the one or more molecular feature values.

### CROSS-REFERENCE TO RELATED APPLICATIONS

The present application is a continuation of PCT/JP2024/034654 filed Sep. 27, 2024, which claims the priority to Japanese Patent Application No. 2023-170464, filed on Sep. 29, 2023, the entire content of which is incorporated herein by reference.

### TECHNICAL FIELD

The present disclosure relates to information processing methods, presentation methods, programs, and manufacturing methods.

### BACKGROUND ART

Conventionally, as design methods for thermal fluid analysis and thermal cycles, there have been computational fluid dynamics (CFD), thermal cycle simulations capable of calculating heat circulation in an entire circuit by arranging heat exchangers, piping, and the like and calculating heat generated in each process, and mechatronics system simulations capable of calculating changes in waste heat and the like by arranging heat exchangers, piping, and the like designed on a computer. However, calculations including macrostructures such as flow paths as in CFD require visualization of mesoscopic behavior including thermophysical properties such as density and enthalpy, behaviors including phase changes such as gas phase and liquid phase, transient phenomena such as dissolution, and such dynamic processes, and appropriate modeling of part or all thereof.

As a method for grasping such mesoscopic behavior, Gibbs Ensemble Monte Carlo Simulation (GEMC) has been used, as described in Non-Patent Document 1. This method obtains an expected value of a physical quantity in statistical mechanics by numerical integration using random numbers in order to realize a Boltzmann distribution. However, since this method is applicable only in an equilibrium state, it is difficult to calculate quasi-static and dynamic processes that change over time, which are essential for thermal cycles and thermal fluid analysis.

On the other hand, as a method for analyzing nonequilibrium behavior of atoms and molecules, there is a molecular dynamics (MD) method. In the molecular dynamics method, it is necessary to set characteristics of atoms and molecules using various potential parameters called force fields. Conventionally, there has been a method for setting Lennard-Jones potential parameters among potential parameters, as in Patent Document 1.

### PRIOR ART DOCUMENTS

### Non-Patent Documents

Non-Patent Document 1: The Journal of Physical Chemistry B, (US), 2010, Vol. 114, Issue 31, p. 10133-10142

### PATENT DOCUMENTS

Patent Document 1: JP 2021-005344 A

### SUMMARY OF THE INVENTION

### Problems to be Solved by the Invention

However, only some of the many potential parameters included in the MD method are automatically determined, and experimental values obtained from experimental results, prior literature, and the like are still always required; therefore, this cannot necessarily be said to be an effective means.

The present disclosure provides an information processing method, a presentation method, a program, and a manufacturing method that enable calculation of a change in a physical property value of a substance without experimental values with respect to molecules constituting the substance.

### Solutions to the Problems

An information processing method according to one aspect of the present disclosure is an information processing method executed by a computer system, and includes: acquiring one or more chemical formulas of respective one or more types of molecules constituting a substance and a composition ratio of the one or more types of molecules; calculating, from the one or more chemical formulas of the respective one or more types of molecules, one or more molecular feature values of the respective one or more types of molecules; and generating physical property change data representing a change in a physical property value of the substance by using the composition ratio and the one or more molecular feature values.

A presentation method according to one aspect of the present disclosure is a presentation method for thermal fluid analysis, executed by a computer system, and includes: receiving input of one or more chemical formulas of respective one or more molecules constituting a substance and a composition ratio of the one or more molecules; generating an image indicating continuous data for each physical property value required for thermal fluid analysis based on the one or more chemical formulas and the composition ratio which are input; and presenting the image generated.

A program according to one aspect of the present disclosure is a program for causing a computer system to execute the above information processing method.

A manufacturing method according to one aspect of the present disclosure is a manufacturing method for physical property change data of a substance, executed by a computer system, and includes: acquiring one or more chemical formulas of respective one or more molecules constituting a substance and a composition ratio of the one or more molecules; determining, from the one or more chemical formulas of the respective one or more molecules, one or more molecular feature values of the respective one or more molecules; and generating physical property change data representing a change in a physical property value of the substance by using the composition ratio and the one or more molecular feature values.

### Effects of the Invention

Aspects of the present disclosure enable calculation of a change in a physical property value of a substance without experimental values with respect to molecules constituting the substance.

### BRIEF DESCRIPTION OF THE DRAWINGS

FIG. 1 is a block diagram of a computer system that executes an information processing method according to one embodiment.

FIG. 2 is a flowchart of the information processing method according to one embodiment.

FIG. 3 is an illustrative diagram showing an example of a molecular structure.

FIG. 4 is an illustrative diagram of a method for determining Lennard-Jones potential parameters.

FIG. 5 is a graph showing a relationship between an intermolecular distance and potential energy when an orientation angle is changed.

FIG. 6 is a diagram showing an example of a physical structure of a substance.

FIG. 7 is an illustrative diagram of an NPE calculation.

FIG. 8 is a graph showing a method for determining latent heat.

FIG. 9 is an illustrative diagram of forming a continuous function by fitting density with respect to the reciprocal of temperature.

FIG. 10 is an illustrative diagram of forming a continuous function by fitting kinematic viscosity with respect to temperature.

FIG. 11 is an illustrative diagram of forming a continuous function by fitting vapor pressure with respect to temperature.

FIG. 12 is an illustrative diagram showing an example of a presentation screen for presenting physical property change data.

FIG. 13 is an illustrative diagram of a gas distribution in thermal fluid analysis according to Example 1.

FIG. 14 is an illustrative diagram of a flow velocity distribution in the thermal fluid analysis according to Example 1.

FIG. 15 is an illustrative diagram of a pressure distribution in the thermal fluid analysis according to Example 1.

FIG. 16 is an illustrative diagram of a temperature distribution in the thermal fluid analysis according to Example 1.

FIG. 17 is a block diagram of a computer system that executes an information processing method according to one variation.

### DETAILED DESCRIPTION

### 1. Embodiments

### 1.1 Configurations

An information processing method according to the present embodiment is a method executed by a computer system for calculating a change in a physical property value of a substance. The information processing method according to the present embodiment enables prediction of a physical change of a substance. The information processing method according to the present embodiment can be applied to simulation of a thermal fluid cycle in a geometric configuration, computational fluid dynamics, mechatronics system simulation, or thermal fluid analysis.

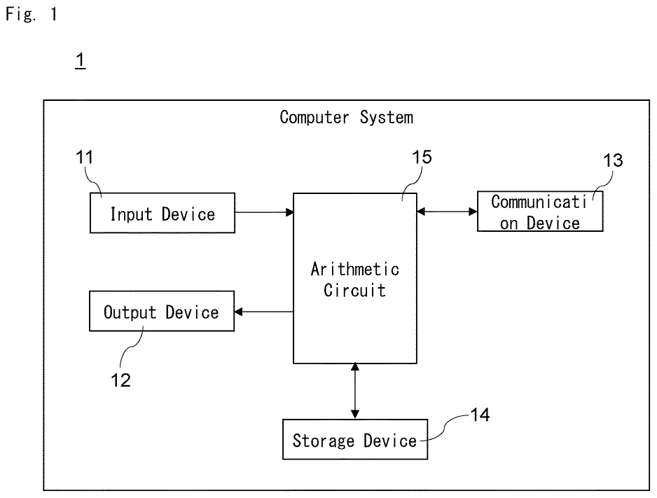

FIG. 1 is a block diagram of a computer system 1 configured to perform an information processing method. The computer system 1 includes an input device 11, an output device 12, a communication device 13, a storage device 14, and an arithmetic circuit 15.

The input device 11 includes one or more human-machine interfaces for inputting information. Examples of human-machine interfaces include input interfaces such as a keyboard, pointing devices (e.g., mouse, trackball), touchpad, or positional input devices of a touch panel display. One or more human-machine interfaces of the input device 11 may be a built-in type or an external type.

The output device 12 includes one or more human-machine interfaces for outputting information. Examples of human-machine interfaces include output interfaces such as a display, speaker, or display devices of a touch panel display. One or more human-machine interfaces of the output device 12 may be a built-in type or an external type.

The communication device 13 is connectable to a communication network and has a function to communicate via the communication network. The communication device 13 conforms to a predetermined communication protocol. The predetermined communication protocol may be selected from various known wired and wireless communication standards.

The storage device 14 is used to store information used a computer system or information generated by the computer system 1. The storage device 14 includes one or more storages (non-transitory storage media). The storage may be, for example, any of a hard disk drive, an optical drive, and a solid-state drive (SSD). The storage may be any of a built-in type, an external type, and a network-attached storage (NAS) type.

The arithmetic circuit 15 is connected to the input device 11, the output device 12, and the communication device 13, and is accessible to the storage device 14. The arithmetic circuit 15 includes one or more processors (microprocessors) and one or more memories. By executing a program (stored in one or more memories or in the storage device), the one or more processors realize various functions. The program may be pre-recorded in the storage device, or may be provided via an electric communication line such as the Internet, or recorded on a non-transitory recording medium such as a memory card.

In the present embodiment, the computer system 1 is implemented by a single computer. Examples of the computer include a server, a personal computer (desktop computer, laptop computer), and a mobile terminal (smartphone, tablet terminal, etc.).

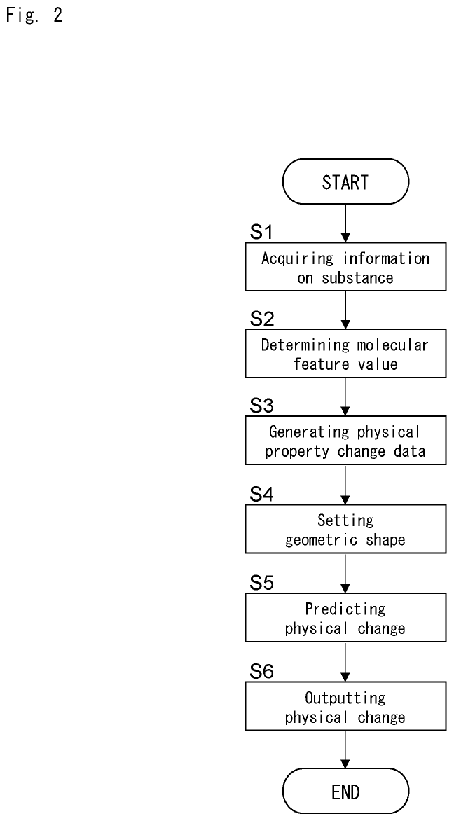

The information processing method according to the present embodiment is implemented by the computer system 1 executing a program. FIG. 2 is a flowchart of the information processing method according to the present embodiment.

The information processing method includes an acquisition step S1, a molecular feature value determination step S2, a physical property change data generation step S3, a geometric shape condition setting step S4, a physical change prediction step S5, and a physical change output step S6.

The acquisition step S1 acquires information on a substance. The information on the substance includes one or more chemical formulas of respective one or more molecules constituting the substance and a composition ratio of the one or more molecules. The chemical formula may be selected from one or more of an empirical formula (elemental composition formula, compositional formula), a molecular formula, a structural formula, and a rational formula. In the present embodiment, the chemical formula is a structural formula. A structural formula is preferable because it not only indicates the number of atoms of each molecule but also represents how the atoms constitute the molecule and explicitly or implicitly indicates chemical bonds between the atoms, thereby facilitating identification of the compound. The composition ratio is a ratio of the one or more molecules constituting the substance. The substance may be a pure substance or a mixture. When the substance is a pure substance, the substance is composed of only one type of molecule, and naturally, the composition ratio is 100% of that one type of molecule. When the substance is a mixture, the substance is composed of a plurality of types of molecules, and the composition ratio indicates a molar ratio or a mass ratio of each molecule. As one example, a substance X is specified as containing two types of molecules, molecule A and molecule B, at a molar composition ratio of C:D.

The molecular feature value determination step S2 determines molecular feature values of the respective one or more molecules from the chemical formulas of the respective one or more molecules.

The molecular feature value indicates molecular-level characteristics of the substance. The molecular feature value is determined from first principles by quantum chemical calculations based on the chemical formulas or structural formulas of the respective one or more molecules of the substance acquired in the acquisition step S1. Examples of quantum chemical calculation methods include Density Functional Theory (DFT), Coupled Cluster (CC), and Multireference Configuration Interaction (MRCI).

The molecular feature value includes a molecular structure and a molecular force field.

The molecular structure is determined, for example, based on a most stable structure obtained by quantum chemical calculation from the chemical formula of each molecule. The most stable structure is determined based on a structure optimization calculation. That is, the most stable structure of a molecule can be determined by solving a minimization problem on a potential energy surface. As one example, the potential energy may be determined using, for example, a Coupled Cluster (CC) method, which is an ab initio molecular orbital method, and a correlation-consistent basis set to which polarization functions are added as basis functions.

In the present embodiment, the molecular structure includes at least one of a bond length, a bond angle, or a dihedral angle. The bond length is a distance between two atoms in a molecule. The bond angle is an angle between bond axes extending from one atom to two atoms when the two atoms are bonded to the one atom in the molecule. The dihedral angle is an angle between two planes passing through two sets of three atoms that share two atoms. The bond length, the bond angle, or the dihedral angle can be determined from the most stable structure. The bond length, the bond angle, or the dihedral angle may be determined for each combination of atoms for which these can be defined.

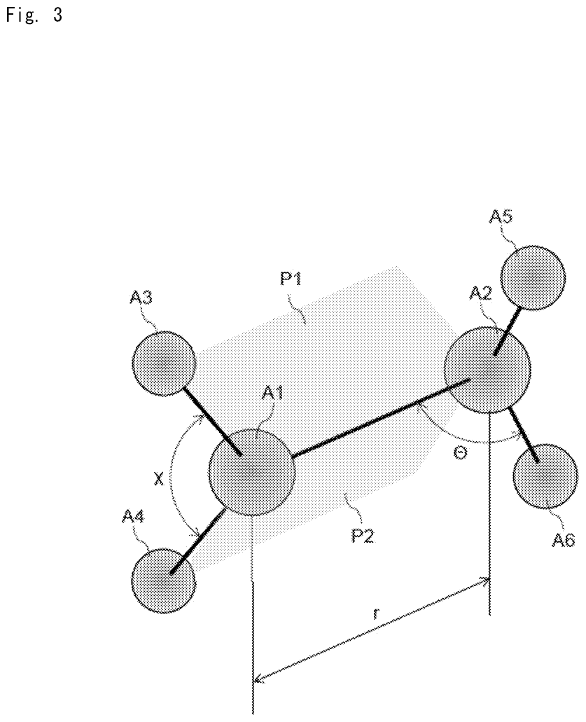

FIG. 3 is an illustrative diagram showing an example of a molecular structure. The molecular structure shown in FIG. 3 has atoms A1 to A6. The atom A1 is bonded to the atom A2, the two atoms A3 and A4 are bonded to the atom A1, and the two atoms A5 and A6 are bonded to the atom A2. The bonding relationships among the atoms A1 to A6 can be determined from the chemical formula of the molecule.

For the molecular structure shown in FIG. 3, as bond lengths, a bond length between the atoms A1 and A2, a bond length between the atoms A1 and A3, a bond length between the atoms A1 and A4, a bond length between the atoms A2 and A5, and a bond length between the atoms A2 and A6 are determined. As bond angles, a bond angle of the atoms A3, A1, and A4, a bond angle of the atoms A2, A1, and A3, a bond angle of the atoms A2, A1, and A4, a bond angle of the atoms A5, A2, and A6, a bond angle of the atoms A1, A2, and A5, and a bond angle of the atoms A1, A2, and A6 are determined. As dihedral angles, a dihedral angle between a plane defined by the atoms A1, A2, and A3 and a plane defined by the atoms A1, A2, and A4; a dihedral angle between a plane defined by the atoms A1, A2, and A3 and a plane defined by the atoms A1, A2, and A5; a dihedral angle between a plane defined by the atoms A1, A2, and A3 and a plane defined by the atoms A1, A2, and A6; a dihedral angle between a plane defined by the atoms A1, A2, and A4 and a plane defined by the atoms A1, A2, and A5; a dihedral angle between a plane defined by the atoms A1, A2, and A4 and a plane defined by the atoms A1, A2, and A6; and a dihedral angle between a plane defined by the atoms A1, A2, and A5 and a plane defined by the atoms A1, A2, and A6 are determined.

In FIG. 3, r indicates a bond length between the atoms A1 and A2. Θ indicates a bond angle of the atoms A1, A2, and A6. χ indicates a dihedral angle between a plane defined by the atoms A1, A2, and A3 and a plane defined by the atoms A1, A2, and A4, that is, an angle between a plane P1 passing through the three atoms A1, A2, and A3 and a plane P2 passing through the three atoms A1, A2, and A4.

The molecular force field is determined based on the molecular structure. The molecular force field includes at least one of an intramolecular force field or an intermolecular force field (intermolecular interaction).

The intramolecular force field includes a force constant for at least one of a bond length, a bond angle, or a dihedral angle. The force constant for a bond length, a bond angle, or a dihedral angle is determined using the most stable structure, for example, by normal mode analysis. In the present embodiment, the force constant for a bond length, a bond angle, or a dihedral angle may be determined by performing normal mode analysis on the most stable structure.

In determining the force constant for a bond length, potential energy is calculated multiple times by expanding or contracting the bond length from that of the most stable structure. The potential energy may be determined using, for example, a Coupled Cluster method, which is an ab initio molecular orbital method, and a correlation-consistent basis set to which polarization functions are added as basis functions. From the obtained relationship between the potential energy and the bond length, the force constant for the bond length can be determined. For example, the force constant for the bond length can be determined by fitting the relationship between the potential energy and the bond length with the following equation (1). In the following equation (1), Eᵣ denotes the potential energy, r denotes the bond length, r₀ denotes the bond length of the most stable structure, and kᵣ denotes the force constant for the bond length.

$$\begin{matrix}[ Math . 1 ] & \\ {E}_{r} = \frac{1}{2} {{k}_{r} ( r - {r}_{0} )}^{2} & ( 1 )\end{matrix}$$

In determining the force constant for a bond angle, the potential energy is calculated multiple times while increasing or decreasing the bond angle from that of the most stable structure by a predetermined angle (for example, 5 degrees). The method for calculating the potential energy may be the same as described above. From the obtained relationship between the potential energy and the bond angle, the force constant for the bond angle can be determined. For example, the force constant for the bond angle can be determined by fitting the relationship between the potential energy and the bond angle with the following equation (2). In the following equation (2), E_{Θ} denotes the potential energy, Θ denotes the molecular angle, Θ₀ denotes the molecular angle of the most stable structure, and k_{Θ} denotes the force constant for the molecular angle.

$$\begin{matrix}[ Math . 2 ] & \\ {E}_{Θ} = \frac{1}{2} {{k}_{Θ} ( Θ - {Θ}_{0} )}^{2} & ( 2 )\end{matrix}$$

In determining the force constant for a dihedral angle, the potential energy is calculated multiple times while changing the phase from the dihedral angle of the most stable structure. The method for calculating the potential energy may be the same as described above. From the obtained relationship between the potential energy and the dihedral angle, the force constant for the dihedral angle can be determined. For example, the force constant for the dihedral angle can be determined by fitting the relationship between the potential energy and the dihedral angle with the following equation (3). In the following equation (3), E_{χ} denotes the potential energy, χ denotes the dihedral angle, n denotes the number of planes, δ denotes the phase, and k_{χ} denotes the force constant for the dihedral angle.

$$\begin{matrix}[ Math . 3 ] & \\ {E}_{χ} = {k}_{χ} [ 1 + cos ( n χ - δ ) ] & ( 3 )\end{matrix}$$

The intermolecular force field may be determined by charge distribution analysis. The intermolecular force field includes at least one of a molecular charge or a van der Waals potential parameter. The molecular charge is determined so as to reproduce an electrostatic potential of the most stable structure. For determining the molecular charge, Mulliken charges, RESP charges, NBO charges, AM1-BCC charges, and the like may be used. In particular, RESP charges are preferable because they assume a Coulomb potential in molecular dynamics. The van der Waals potential parameter may be determined using, for example, a Lennard-Jones potential or a Buckingham potential. The following equation (4) shows a general form of the Lennard-Jones potential.

$$\begin{matrix}[ Math . 4 ] & \\ {V}_{LJ} ( {r}_{i j} ) = 4 {ϵ}_{i j} ( {( \frac{{σ}_{i j}}{{r}_{i j}} )}^{q} - {( \frac{{σ}_{i j}}{{r}_{i j}} )}^{p} ) & ( 4 )\end{matrix}$$

In the above equation (4), V_{LJ} denotes a Lennard-Jones potential, rᵢⱼ denotes an intermolecular distance, εᵢⱼ and σᵢⱼ denote Lennard-Jones potential parameters, p denotes an order of an attractive term, and q denotes an order of a repulsive term. Various forms exist for the orders (p, q), such as a (6, 12) potential and a (6, 9) potential type; however, from the viewpoints of ease of handling values and expandability to molecular dynamics calculation software, (p, q)=(6, 12) is more preferable.

The Lennard-Jones potential parameters εᵢⱼ and σᵢⱼ required for calculation of the Lennard-Jones potential are also obtained by quantum chemical calculation.

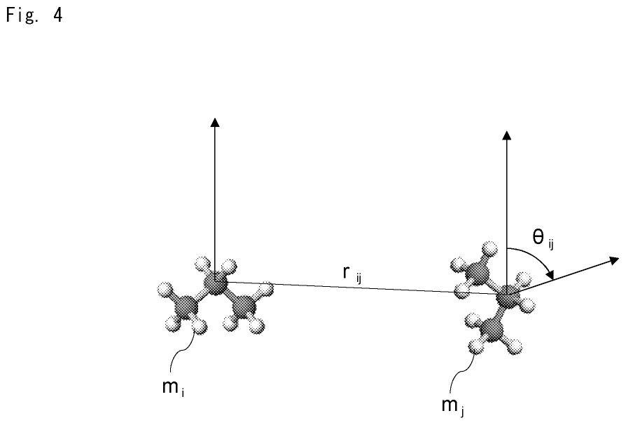

FIG. 4 is an illustrative diagram of a method for determining Lennard-Jones potential parameters. Specifically, for example, as shown in FIG. 4, two identical molecules mᵢ and mⱼ are prepared, and by quantum chemical calculation, the distance (intermolecular distance) rᵢⱼ between molecules and the intermolecular orientation angle θᵢⱼ at which the potential energy becomes a most stable point are determined while fixing molecular structures.

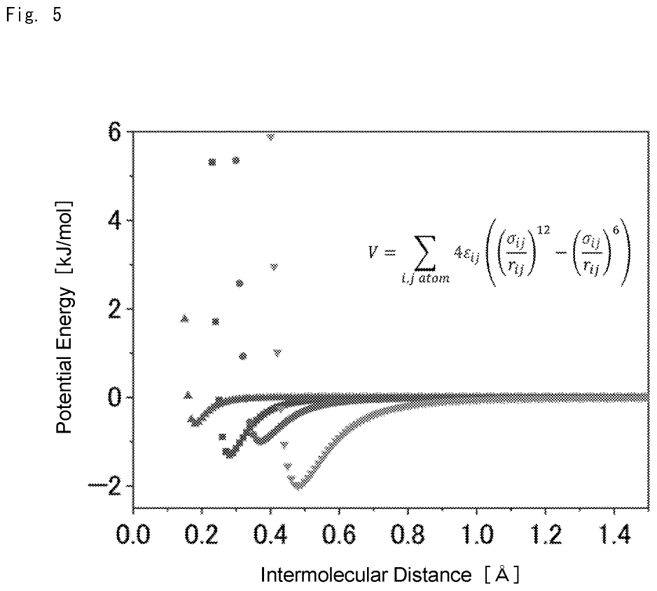

FIG. 5 is a graph showing a relationship between the intermolecular distance rᵢⱼ and potential energy when the orientation angle θᵢⱼ is changed. In FIG. 5, “▴” indicates a case where the orientation angle θᵢⱼ is 90°, “▪” indicates a case where the orientation angle θᵢⱼ is 60°, “●” indicates a case where the orientation angle θᵢⱼ is 30°, and “▾” “indicates a case where the orientation angle θᵢⱼ is 0°.

While changing the intermolecular distance rᵢⱼ and/or the intermolecular orientation angle θᵢⱼ around the most stable point, the potential energy is calculated multiple times, and a Lennard-Jones potential V_{LJ} with respect to the intermolecular distance rᵢⱼ is calculated. The method for calculating the potential energy may be the same as described above. The Lennard-Jones potential V_{LJ} is calculated at a plurality of intermolecular distances rᵢⱼ, and the Lennard-Jones potential parameters εᵢⱼ and θᵢⱼ are determined so as to satisfy the relationship between the Lennard-Jones potential V_{LJ} and the intermolecular distance rᵢⱼ shown in the above equation (4). Arranging three or more identical molecules is more preferable because a more precise Lennard-Jones potential can be obtained, and thus accuracy of the parameters may be improved.

In this manner, in the molecular feature value determination step S2, from the chemical formulas of the respective one or more molecules, a molecular feature value including a molecular structure including a bond length, a bond angle, and a dihedral angle of the respective one or more molecules, and a molecular force field including charges and van der Waals potential parameters, are determined from first principles by quantum chemical calculation without using experimental values. Therefore, even without experimental values of the one or more molecules constituting the substance, the molecular feature values of the respective one or more molecules can be determined.

The physical property change data generation step S3 determines a physical property value (physical state) of a substance based on the one or more chemical formulas and the composition ratio of the respective molecules. More specifically, in the physical property change data generation step S3, a physical structure of the substance is obtained by molecular dynamics simulation from the molecular feature value determined in the molecular feature value determination step S2 and the composition ratio of the one or more molecules acquired in the acquisition step S1, and the physical property value of the substance are calculated by statistical mechanics calculations using the physical structure of the substance calculated by the molecular dynamics simulation. The calculated discrete physical property values are rewritten into continuous functions based on external input values such as temperature and pressure, thereby generating physical property change data.

The physical structure of the substance includes at least one of a bulk structure, molecular orientation, a micro-aggregate structure, or phase separation. In determining an initial state of the physical structure of the substance, a bulk structure in which one or more molecules of the substance are randomly arranged using a molecular dynamics method described later may be used. The initial structure of the bulk structure of the substance may be determined, for example, by performing a stable structure calculation based on the composition ratio and the molecular feature values. As one example, a bulk initial calculation structure of the substance may be obtained by setting the number of molecules of the substance to a desired value, randomly arranging the molecules of the substance, and then performing a stable structure calculation. The physical structure of the substance can be used to determine the physical property values of the substance.

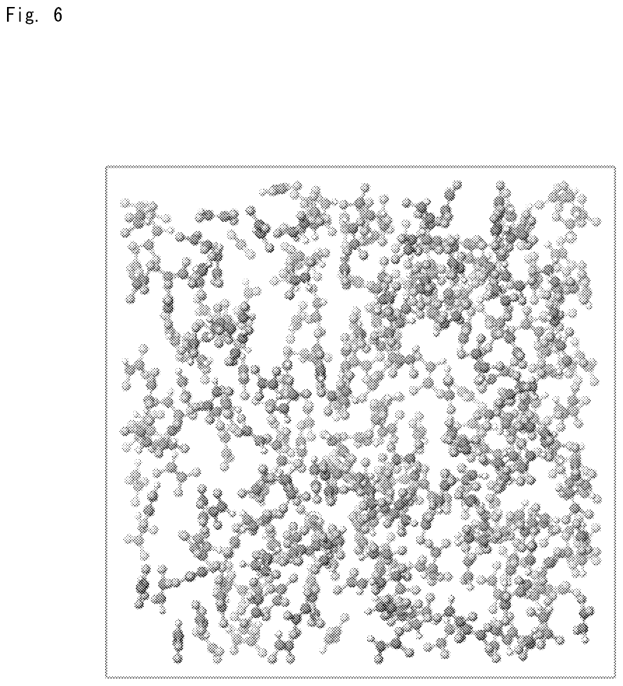

FIG. 6 is a diagram showing an example of a physical structure of a substance. As shown in FIG. 6, the physical structure of the substance is provided as a three-dimensional display such as arrangement coordinates or an image of each molecule.

Examples of the molecular dynamics simulation include an NPT simulation in which pressure and temperature are kept constant with respect to an initial structure of a bulk structure, an NVT simulation in which volume and temperature are kept constant with respect to the initial structure of the bulk structure, an NVE simulation in which volume and energy are kept constant with respect to the initial structure of the bulk structure, an NPE simulation in which pressure and internal energy are kept constant with respect to the initial structure of the bulk structure, a BAR calculation method capable of calculating a solvation Gibbs free energy and an excess Gibbs free energy of the substance, and an ER calculation method.

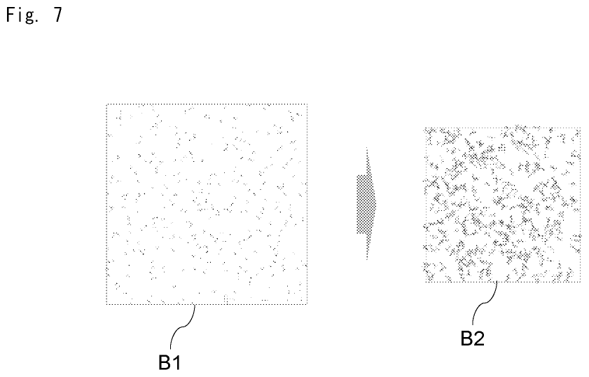

FIG. 7 is an illustrative diagram of an NPE simulation. In the NPE simulation, by performing a simulation on an initial structure B1 of a bulk structure under a condition that pressure and internal energy are constant, a structure B2 after change of the bulk structure can be obtained. The structure B2 after change obtained in this manner can be used for calculation of the physical property values of the substance.

In the molecular dynamics simulation, it is necessary to arrange a large number of molecules in a certain compartment (cell) for calculation. The number of molecules arranged in the compartment is preferably 200 or more, and more preferably 500 or more. When the number of molecules is 200 or more, intermolecular interactions can be calculated, and when the number is 500 or more, intermolecular interactions can be stably calculated and potentials can be stably obtained by arbitrary NPT simulation, NVT simulation, NVE simulation, NPE simulation, BAR calculation, and ER calculation. The physical structure of the substance obtained by such calculations may be used as time-series data, or, for example, structural data and data groups at a certain time may be extracted and used for subsequent statistical mechanics calculations.

The physical property values of the substance are obtained by performing statistical mechanics calculations based on the obtained physical structure of the substance. For example, the physical property values include at least one of density, vapor pressure (saturated vapor pressure), kinematic viscosity, diffusion coefficient, latent heat (vaporization/liquefaction), (specific) enthalpy, thermal conductivity, isochoric heat capacity, isobaric heat capacity, excess Gibbs free energy, solvation Gibbs free energy, and Gibbs free energy. Among these, through equilibrium processes, density, vapor pressure (saturated vapor pressure), kinematic viscosity, diffusion coefficient, latent heat (vaporization/liquefaction), (specific) enthalpy, thermal conductivity, isochoric heat capacity, isobaric heat capacity, and boiling point and dew point of a pure substance are obtained. Among these, physical property values obtained in an equilibrium state in a system are density, vapor pressure (saturated vapor pressure), latent heat (vaporization/liquefaction), and (specific) enthalpy. On the other hand, physical property values obtained in a nonequilibrium process within a system or between physical structures of a plurality of substances are kinematic viscosity, diffusion coefficient, thermal conductivity, isochoric heat capacity, isobaric heat capacity, excess Gibbs free energy, solvation Gibbs free energy, and Gibbs free energy. However, the physical property values of the substance are calculated as discrete values associated with temperature and pressure.

The NPT simulation is a calculation method in which molecular dynamics simulation is performed by applying conditions of the number of molecules, pressure, and temperature. For the obtained physical structure state of the substance, it can be used, for example, to determine density, (specific) enthalpy, isobaric heat capacity, latent heat, diffusion coefficient, and excess Gibbs free energy. The NVT simulation can be used, for example, to determine vapor pressure, isochoric heat capacity, and densities of two layers in a gas-liquid mixed state.

More specifically, in the case of density, it is obtained by an ensemble average with respect to calculation time of a volume of the calculated molecular structure relative to the number of molecules incorporated into the molecular dynamics simulation.

In the case of (specific) enthalpy, it can be calculated by an ensemble average with respect to calculation time of a sum of internal energy U, which is a sum of potential energy and kinetic energy due to intramolecular and intermolecular interactions based on the molecular force field, and a product pV of the applied pressure and the volume of the molecular structure obtained by calculation.

In the case of isobaric heat capacity, it can be calculated from a change in specific enthalpy when temperature is slightly changed under a constant pressure condition in the NPT simulation. More specifically, a minute difference in specific enthalpy during calculation of a physical structure state and a temperature difference at that point are recorded, and an ensemble average with respect to calculation time of a result obtained by dividing the minute difference in specific enthalpy by the temperature difference is calculated.

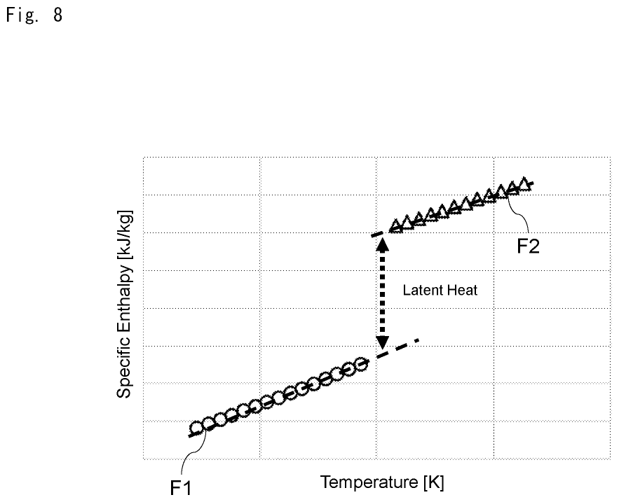

In the case of latent heat, an NPT simulation is performed while fixing pressure to a desired value and sequentially changing only temperature to obtain specific enthalpy. FIG. 8 is a graph showing a method for determining latent heat. As shown in FIG. 8, in a graph in which a horizontal axis represents temperature and a vertical axis represents specific enthalpy, a difference in specific enthalpy between a high-density state F1 and a low-density state F2 is defined and calculated as latent heat.

In the case of diffusion coefficient, a mean square displacement of molecules during the NPT simulation is output with respect to time, and the diffusion coefficient can be calculated from an ensemble average of a slope thereof. The calculation time required for these ensemble averages is preferably 1 ns or more in order to stabilize numerical results, and more preferably 5 ns or more for further stabilization of numerical values.

The excess Gibbs free energy may be calculated using the following method. A physical structure of N molecules and one target molecule is obtained by molecular dynamics simulation, and an energy change caused by adding one target molecule into a gap of the physical structure of the N molecules is estimated. This is repeated M times to obtain an ensemble average with respect to the energy when one target molecule is added to the N molecules. On the other hand, molecular dynamics simulation is performed in advance for N+1 molecules obtained by adding one target molecule to the N molecules to calculate a physical structure and energy of the N+1 molecules. In practice, when one target molecule is added to the N molecules, the surrounding N molecules are rearranged so as to become stable, and therefore an energy of the physical structure of the N+1 molecules should be lower. Accordingly, the excess Gibbs free energy is energy obtained by subtracting the energy when one target molecule is added into a gap of the N molecules from the energy of the physical structure of the N+1 molecules. Further, the Gibbs free energy is obtained by adding a Gibbs free energy of an ideal gas to the excess Gibbs free energy. The Gibbs free energy of the ideal gas can be readily calculated statistically.

The NVE simulation is a calculation method in which molecular dynamics simulation is performed by applying conditions of the number of molecules, volume, and temperature, and can be used to determine thermophysical properties in an adiabatic process, such as entropy, by performing statistical mechanics calculations on the obtained physical structure state of the substance.

The NPT simulation, NVT simulation, NVE simulation, NPE simulation, BAR calculation, or ER calculation may be used in combination in order to simulate a desired state or a desired process of the substance. For example, in order to obtain an appropriate bulk structure or to perform a main calculation for obtaining physical property values after annealing of a bulk structure, the NVT simulation, NPT simulation, NVE simulation, NPE simulation, BAR calculation, or ER calculation may be appropriately combined and used.

As described above, by performing statistical mechanics calculations using the substance structure determined by molecular dynamics simulation, it is possible to obtain a kinematic viscosity, density, latent heat, (specific) enthalpy, thermal conductivity, isochoric heat capacity, isobaric heat capacity, diffusion coefficient, vapor pressure, (specific) enthalpy change due to adiabatic compression, boiling point, dew point, excess Gibbs free energy, solvation Gibbs free energy, or Gibbs free energy.

As described above, by performing statistical mechanics calculations using the substance structure determined by molecular dynamics simulation, physical property values of the substance can be calculated. Generally, characteristic values of a substance are obtained for each substance structure. That is, physical property values are generally obtained as a list of fragmented or discretized physical property values. In some cases, such physical property values are difficult to apply to simulation of a thermal fluid cycle, computational fluid dynamics, mechatronics system simulation, or thermal fluid analysis. In simulation of a thermal fluid cycle and the like, values of physical states corresponding to various conditions are required. For example, in computational fluid dynamics, temperature may change continuously. When only a list of physical property values is available, a value of a physical state corresponding to a required temperature may not exist in the list, and it is necessary to separately consider handling of such non-existent values. In computational fluid dynamics, temperature may also be regarded as substantially constant in some cases. The thermal fluid in the present embodiment is applicable to both fluid calculations in which temperature changes and those in which temperature is fixed.

Accordingly, for the physical property values, a plurality of values (discrete values) obtained with respect to temperature and pressure may be acquired so that the physical property change data includes a continuous function with respect to temperature and pressure. In such continuous functionalization of the physical property change data, for example, conversion of discrete values of physical property values into a continuous function may be performed by interpolation between discrete values or approximation by fitting, or may be performed using separate software. For example, by using machine learning such as deep learning, symbolic regression, or Bayesian regression, or methods such as a least squares method, it becomes possible to convert physical property values into an appropriate continuous function based on discrete values of the physical property values. By using such continuous functionalization processing, it becomes possible to output physical property values corresponding to various inputs.

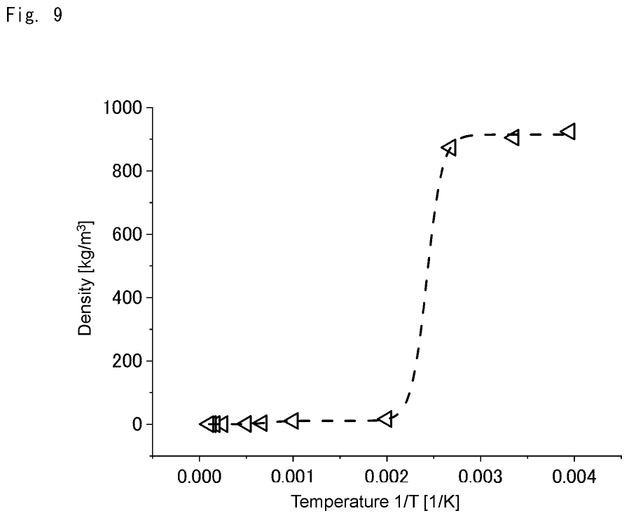

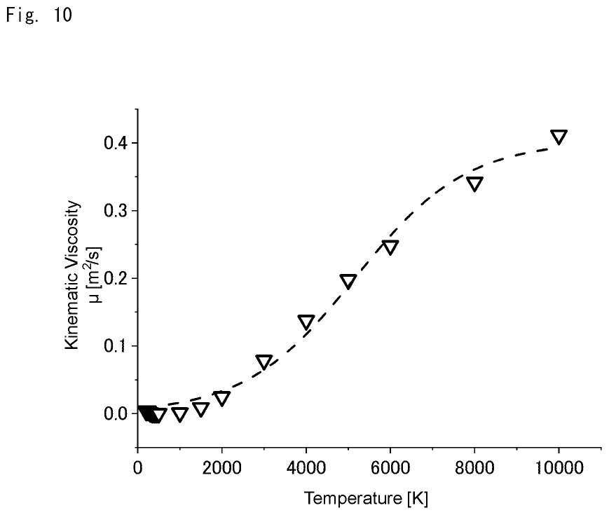

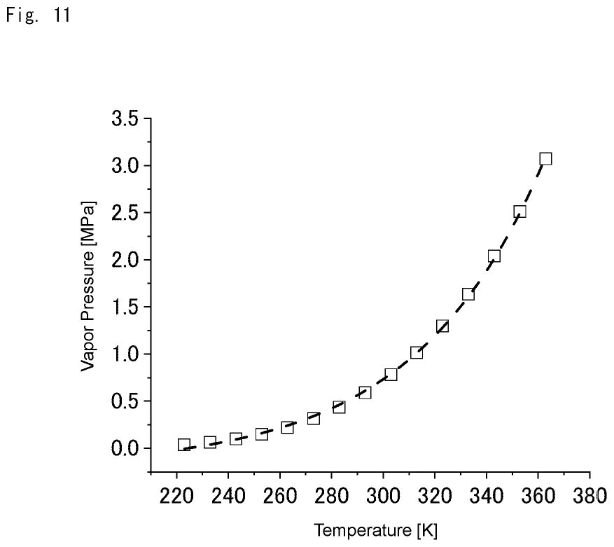

Next, an example of continuous functionalization will be described with reference to FIG. 9 to FIG. 11. FIG. 9 is an illustrative diagram of forming a continuous function by fitting density with respect to the reciprocal of temperature. As shown in FIG. 9, in the case of density, density is plotted for calculation points obtained by changing temperature conditions with respect to the reciprocal of a certain temperature. A fitting function can be defined so as to satisfy these plotted points. Next, while sequentially changing pressure, a graph of temperature and density is drawn to obtain parameter changes of the previously determined fitting function. By applying a fitting function to a graph in which the pressures used in the calculation are set on the x-axis and the obtained parameters of the fitting function are set on the y-axis, the fitting parameters can be defined by temperature and pressure, thereby achieving continuous functionalization. FIG. 10 is an illustrative diagram of forming a continuous function by fitting a kinematic viscosity with respect to temperature. FIG. 11 is an illustrative diagram of forming a continuous function by fitting vapor pressure with respect to temperature. The kinematic viscosity and vapor pressure can also be converted into continuous functions by the same method as described above.

As described above, in the present embodiment, the physical property change data generation step S3 includes a step of obtaining discrete physical property values by performing statistical mechanics calculations using the substance structure determined by molecular dynamics simulation, and a step of converting the physical property values into a continuous function based on the discrete physical property values. Thus, physical property change data including continuously functionalized physical property values can be generated.

The computer system 1 may include a program capable of presenting (outputting) the physical property change data obtained in the physical property change data generation step S3. More specifically, by inputting a name or chemical formula of a substance and a composition ratio of the substance into software, molecular feature values are calculated based on quantum chemical calculations according to the input name or chemical formula and composition ratio, physical property values required for thermal fluid calculation are calculated by molecular dynamics simulation and statistical mechanics calculation from the composition ratio and the molecular feature values, a library or image of continuous data representing changes in the calculated physical property values is generated, and simulation of a thermal fluid cycle, computational fluid dynamics, or mechatronics system simulation may be executed using the generated library or image.

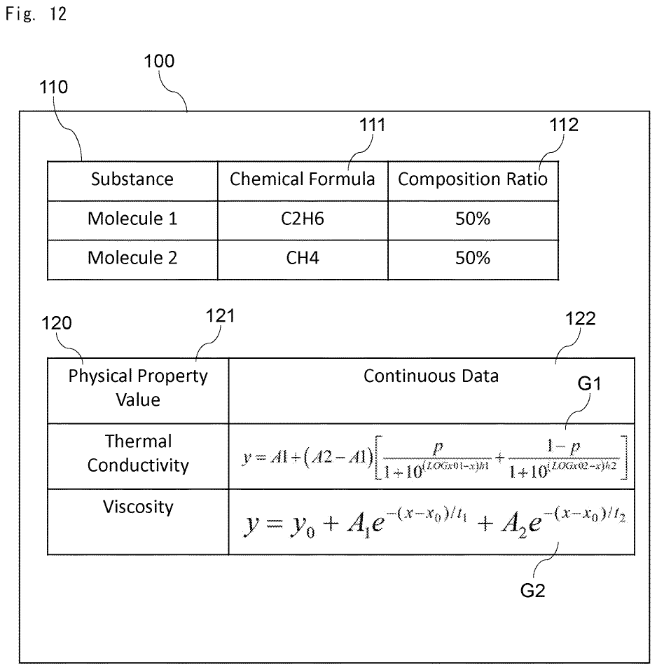

FIG. 12 is an illustrative diagram showing an example of a presentation screen 100 for presenting physical property change data.

The presentation screen 100 includes an input area 110 and an output area 120.

The input area 110 is used to input information on a substance for which physical property values are to be calculated. The input area 110 includes a first area 111 and a second area 112.

The first area 111 is an area for inputting chemical formulas of respective one or more molecules constituting the substance. The first area 111 enables input of arbitrary chemical formulas. FIG. 12 shows a state in which a chemical formula C₂H₆ representing ethane is input as molecule 1 constituting the substance, and a chemical formula CH₄ representing methane is input as molecule 2 constituting the substance. The first area 111 may allow input of a name instead of a chemical formula. In this case, a table associating names with chemical formulas is prepared in advance.

The second area 112 is an area for inputting a composition ratio of the one or more molecules. The second area 112 enables input of arbitrary numerical values. FIG. 12 shows a state in which 50% is input as the composition ratio of molecule 1 and 50% is input as the composition ratio of molecule 2.

The output area 120 includes a description area 121 and a presentation area 122.

The description area 121 displays character strings indicating names of physical property values. In FIG. 12, thermal conductivity and viscosity are displayed as the physical property values.

The presentation area 122 presents images G1 and G2 showing continuous data of physical property values calculated based on the information on the substance input to the input area 110. In FIG. 12, the image G1 shows a continuous function of thermal conductivity, and the image G2 shows a continuous function of viscosity. The images G1 and G2 may show graphs as continuous data of the corresponding physical property values. The images G1 and G2 may be either still images or moving images.

As described above, the computer system 1 may include a program for executing a presentation method for thermal fluid calculation. This presentation method receives input of one or more chemical formulas of respective one or more molecules constituting a substance and a composition ratio of the one or more molecules, generates images indicating continuous data for each physical property value required for thermal fluid calculation based on the input chemical formulas and composition ratio, and presents the generated images.

The geometric shape condition setting step S4 sets geometric shape conditions. The geometric shape conditions are used for prediction of a physical change in the physical change prediction step S5. The geometric shape may be one-dimensional, two-dimensional, or three-dimensional. The geometric shape may be configured by arbitrary shapes and combinations thereof, such as a cylinder, a sphere, a quadrangular prism, a pentagonal prism, a hexagonal prism, or an n-gonal prism, and a circle, a quadrilateral, a pentagon, a hexagon, or an n-gon. Further, within the geometric shape, a wall surface that blocks permeation or flow velocity of a component, a permeable membrane that allows passage only in one direction, an inflow portion through which a component can flow in, or an outflow portion through which a component is discharged to the outside of a system may be arbitrarily arranged.

In the physical change prediction step S5, in order to use the Navier-Stokes equations, the continuity equation, the heat conduction equation, and the diffusion equation described later for a geometric shape, boundary conditions are applied. When examining flow velocity or pressure, for example, a condition of inflow velocity is applied to an inflow portion of the flow velocity. For an outflow portion, a pressure at the outflow portion is determined. For wall surfaces other than the inflow portion and the outflow portion in the geometric shape, a condition that a velocity component is 0 is applied. For a permeable membrane, a condition that a velocity component in a direction of the permeable membrane is 0 or more is applied. When examining temperature, for a heat inflow portion, an amount of heat obtained by multiplying a dot product of a normal-direction component and an inflow velocity by density and a temperature difference at the inflow portion and by a constant-pressure specific heat is applied to the inflow portion. At this time, the constant-pressure specific heat and density may use the physical property values obtained in the physical property change data generation step S3. For an inflow portion of temperature itself rather than heat, a temperature of the inflow portion is set to a desired temperature. For a heat outflow portion, a gradient of temperature with respect to a normal direction component at the outflow portion is set to 0. For wall surfaces other than the inflow portion and the outflow portion in the geometric shape, the wall surfaces are set as adiabatic portions, and more specifically, a normal-direction temperature gradient component with respect to the wall surfaces is set to 0. For a permeable membrane, a condition that a temperature gradient in a direction of the permeable membrane is 0 or more is applied. Finally, when examining a concentration of a component, at the inflow portion, a component concentration is set to a desired concentration. At the outflow portion, an inner product of a normal direction component at the outflow portion and a concentration gradient is set to 0. At wall surfaces other than the inflow portion and the outflow portion in the geometric shape, a concentration gradient with respect to a normal component relative to the wall surfaces is set to 0. For a permeable membrane, a condition that a concentration gradient in a direction of the permeable membrane is 0 or more is applied. In this manner, the geometric shape, inflow portion, outflow portion, wall surfaces, permeable membrane, and the like can be set in advance.

The physical change prediction step S5 predicts a physical change of the substance using the physical property change data generated in the physical property change data generation step S3.

Predicting the physical change includes at least one of calculating a change in physical property values used for thermal cycle simulation, computational fluid dynamics, mechatronics system simulation, or thermal fluid analysis in a geometric shape; calculating a change in physical property values for design optimization of thermal fluid cycle simulation, computational fluid dynamics, or mechatronics system simulation in a geometric shape; or calculating a change in physical property values to generate physical property values used for thermal fluid cycle simulation, computational fluid dynamics, or mechatronics system simulation in a geometric shape.

In the physical change prediction step S5, in thermal fluid calculation or thermal cycle simulation, in order to obtain outputs such as pressure distribution, velocity distribution, temperature distribution, and concentration distribution of a substance, input of the physical property values (physical property change data) obtained in the physical property change data generation step S3 and input of the geometric shape conditions set in the geometric shape condition setting step S4 are used.

In the case of computational fluid dynamics calculation, the substance is assumed to be a fluid flowing within a geometric shape, and the Navier-Stokes equations and the continuity equation, which describe motion of velocity or pressure distribution of the fluid, may be used. In the case of a compressible fluid, the Navier-Stokes equations and the continuity equation are respectively expressed by the following equations (5) and (6).

$$\begin{matrix}[ Math . 5 ] & \\ \frac{Dv}{D t} = \frac{1}{ρ} gradp + \frac{μ}{ρ} Δ v + \frac{( χ - \frac{2}{3} ) + μ}{ρ} grad ( ∇ \cdot v ) + \frac{∇ \cdot v}{ρ} grad ( ( χ - \frac{2}{3} ) + μ ) + \frac{1}{ρ} g r a d ( v \cdot grad μ ) + \frac{1}{ρ} r o t ( v \times grad μ ) - \frac{1}{ρ} v Δμ + g & ( 5 )\end{matrix}$$

$$\begin{matrix}[ Math . 6 ] & \\ \frac{\partial ρ}{\partial t} + ∇ \cdot ( ρ v ) = 0 & ( 6 )\end{matrix}$$

In equations (5) and (6), v denotes a velocity of the fluid, ρ denotes a density of the fluid, μ denotes a kinematic viscosity of the fluid, χ denotes a bulk viscosity, and g denotes a gravitational field acting per unit mass of the fluid. Here, as the density ρ and the kinematic viscosity μ of the fluid, the values obtained up to the physical property change data generation step S3 can be used. Since the bulk viscosity χ generally has a very small value, 0 is used. Further, for example, in the case of an incompressible fluid in which density does not change, calculation can be easily performed by setting ∂p/∂t=0 in equation (6). Therefore, the information processing method of the present embodiment is applicable not only to compressible fluids but also to incompressible fluids. By using the Navier-Stokes equations and the continuity equation, which are the two equations described above, two variables, namely the velocity v and the pressure p of the fluid of the substance, can be output, thereby enabling prediction of fluid motion of the substance.

With respect to thermal fluid analysis, an energy conservation equation may be used for temperature change. The energy conservation equation is expressed by the following equation (7).

$$\begin{matrix}\{ Math . 7 ] & \\ ρ {c}_{p} \frac{\partial T}{\partial t} = - ρ {c}_{p} ( v \cdot ∇ T ) + k ( Δ T ) + Q & ( 7 )\end{matrix}$$

In equation (7), T denotes temperature, ρ denotes density, and cₚ denotes isobaric heat capacity, and a value obtained by adding a gas constant R to isochoric heat capacity may also be used instead of heat capacity. Further, k denotes thermal conductivity. Q denotes an amount of heat per unit time.

For example, when an adiabatic change is assumed in which a calculation system undergoes a rapid chemical reaction or state change, such as in case of reaction enthalpy hᵣₑₐ and latent heat Δhₗₐₜ of a substance generated by a chemical reaction, the amount of heat per unit time Q can be expressed by the following equation (8) using Δh_{adi}, which is a result used in the NPE simulation and statistical mechanics calculation of molecular dynamics simulation, as the generated specific enthalpy.

$$\begin{matrix}[ Math . 8 ] & \\ Q = ρ ( {h}_{r e a} + Δ {h}_{l a t} + Δ {h}_{a d i} ) & ( 8 )\end{matrix}$$

However, the latent heat Δhₗₐₜ is an amount of heat generated only when passing through a boiling point or a dew point determined in the structure determination of the substance and the physical property change data generation step S3.

It is assumed that substances A, B, C, and D undergo, for example, a chemical reaction as represented by the following equation (9).

$$\begin{matrix}[ Math . 9 ] & \\ A + B ⇄ C + D & ( 9 )\end{matrix}$$

At this time, for example, for the substances A, B, C, and D, specific enthalpies are defined as h_{A}, h_{B}, h_{C}, and h_{D}, and Gibbs free energies are defined as G_{A}, G_{B}, G_{C}, and G_{D}, respectively. When a reaction Gibbs free energy generated by the chemical reaction of equation (9) is defined as Gᵣₑₐ, Gᵣₑₐ can be expressed as in the following equation (10).

$$\begin{matrix}[ Math . 10 ] & \\ {G}_{r e a} = {G}_{A} + {G}_{B} - {G}_{C} - {G}_{D} & ( 10 )\end{matrix}$$

When the reaction Gibbs free energy Gᵣₑₐ takes a negative value, the reaction proceeds spontaneously. Therefore, when a reaction enthalpy (reaction heat) generated by the chemical reaction is defined as hᵣₑₐ, hᵣₑₐ can be calculated using the following equation (11).

$$\begin{matrix}[ Math . 11 ] & \\ {h}_{r e a} = {h}_{A} + {h}_{B} - {h}_{C} - {h}_{D} & ( 11 )\end{matrix}$$

The reaction enthalpy hᵣₑₐ obtained in this manner is substituted into equation (8) to obtain the amount of heat per unit time Q, and by incorporating this Q into equation (7), a change in temperature distribution caused by reaction heat can be calculated.

As described above, by solving equation (7) using density, isobaric (isochoric) heat capacity, thermal conductivity, specific enthalpy, latent heat, specific enthalpy generated by adiabatic compression, and flow velocity calculated from the Navier-Stokes equations and the continuity equation, which are obtained by molecular dynamics simulation and statistical mechanics calculation, temperature T due to heat transfer of the substance can be output, thereby enabling prediction of a temperature distribution of the substance. Therefore, equation (7) can be solved, and a temperature distribution of the substance with respect to time and space can be calculated.

In order to determine a concentration distribution of a substance, a diffusion equation is used. The diffusion equation is expressed by the following equation (12).

$$\begin{matrix}[ Math . 12 ] & \\ ρ \frac{\partial c}{\partial t} - ∇ \cdot ( ρ D ∇ c + ρ cD \frac{∇ {M}_{n}}{{M}_{n}} ) + ρ ( v \cdot ∇ ) c = {R}_{r e a} & ( 12 )\end{matrix}$$

In equation (12), ρ denotes a density of the substance, c denotes a concentration of the substance, t denotes time, v denotes velocity, D denotes a diffusion coefficient, Mₙ denotes a molecular weight of the substance, and Rᵣₑₐ denotes an amount by which the substance is generated or reduced due to reaction or the like. The density ρ and the diffusion coefficient D use results of structure determination of the substance and calculations up to the physical property change data generation step S3, the velocity v uses results of the Navier-Stokes equations and the continuity equation, and Mₙ can be automatically determined when the chemical formula of the substance acquired in the acquisition step S1 is given. Therefore, equation (12) can be solved, and a concentration distribution of the substance with respect to time and space can be calculated.

Prediction of the physical change is used for thermal fluid cycle simulation, computational fluid dynamics, or mechatronics system simulation by inputting a geometric shape.

The physical change output step S6 outputs a physical change of the substance derived based on the physical property values. More specifically, the physical change output step S6 outputs the physical change predicted in the physical change prediction step S5.

For example, the physical change may be a steady state or a non-steady state including a time-series change of a non-equilibrium phenomenon such as fluid change, heat, or mass transfer. In this case, outputting the physical change includes visualizing, in numerical form or as images, temperature distribution, pressure distribution, velocity distribution, concentration distribution, and different values of physical property values used in calculation, including non-equilibrium processes that change over time. Examples of methods for graphically visualizing such changes in physical property values include moving images and still images. Moving images or still images showing changes in physical property values can also be applied to output to parameters required for models such as nucleation, nucleus growth, or film thickness of a phase-change portion that satisfy the changes in physical property values shown in the moving images or still images, or to acquisition of physical property values by autocorrelation functions. Dynamic visualization such as moving images does not necessarily have to include all states from an initial condition to a state reaching equilibrium.

For example, the physical change may include a change in a physical structure of the substance. In this case, outputting the physical change includes visualizing different states of the physical structure of the substance. The physical structure of the substance can be more easily understood by visualizing phase change or molecular structure. Phase change includes, for example, liquid droplets in a gas phase, a solid phase in a liquid phase, or a liquid phase in a solid phase. A molecular structure refers to a structure in which a plurality of molecules, rather than a single molecule, aggregate and loosely interact to form a structure like a kind of giant molecule. Examples include aggregates of macromolecules such as proteins or surfactants. Quasi-static or dynamic changes such as diffusion of such aggregates or aggregation or dispersion of molecules may be output as moving images or the like. Further, parameters necessary for dispersion/aggregation or diffusion rate and the like may be extracted and quantified from images of the physical structure of the substance using image recognition techniques or the like. Numerical values of parameters obtained in this manner may also be converted into continuous functions.

### 1.2 Application Examples

The information processing method according to the present embodiment described above can be used for visualization of a heat process in a heat exchanger using a substance as a working fluid.

First, information on a substance used as a working fluid is acquired (the acquisition step S1). The information on the substance includes one or more chemical formulas of respective one or more molecules constituting the substance and a composition ratio of the one or more molecules.

Next, molecular feature values of the substance are determined (the molecular feature value determination step S2). Here, the molecular feature values include, for each molecule, a molecular structure and a molecular force field (an intramolecular force field and an intermolecular force field). The molecular structure includes bond lengths, bond angles, and dihedral angles. The molecular force field includes force constants for bond lengths, bond angles, or dihedral angles, RESP charges, and Lennard-Jones potential parameters.

Next, a physical structure of the substance is obtained by molecular dynamics simulation and physical property values are determined by statistical mechanics calculation (the physical property change data generation step S3). The physical property values include heat capacity, thermal conductivity, and specific enthalpy.

As the heat capacity, for example, isobaric heat capacity (isobaric molar heat capacity) or isochoric heat capacity (isochoric molar heat capacity) may be used.

In determining the isobaric heat capacity, molecular dynamics simulation can calculate internal energy, which is a sum of kinetic energy of molecules and intramolecular and intermolecular potentials, based on the calculated physical structure. Therefore, an NPT simulation is performed under desired temperature and pressure conditions, (specific) enthalpy His calculated in principle as a sum of internal energy and a product pV of applied pressure and temperature, and the isobaric heat capacity is calculated from the relational expression of the following equation (13). In equation (13), Cₚ denotes the isobaric heat capacity, H denotes the calculated enthalpy, and T denotes temperature.

$$\begin{matrix}[ Math . 13 ] & \\ {C}_{p} = {( \frac{\partial H}{\partial T} )}_{p} & ( 13 )\end{matrix}$$

In determining the isochoric heat capacity, a canonical ensemble is used. Specifically, an NVT simulation is performed under a certain temperature condition, a temperature difference and a molar internal energy in the system being calculated are obtained, and the isochoric heat capacity is calculated from the relational expression of the following equation (14). In equation (14), Cᵥ denotes the isochoric heat capacity, E denotes energy, and T denotes temperature.

$$\begin{matrix}[ Math . 14 ] & \\ {C}_{v} = {( \frac{\partial E}{\partial T} )}_{v} & ( 14 )\end{matrix}$$

In determining thermal conductivity, an arbitrary NPT simulation is performed, and from a difference between an amount of heat generated in a partial space within the cell and a temperature difference, an ensemble average of an autocorrelation function of a heat flux in the system calculated according to the Green-Kubo formula is obtained, and a differential value with respect to the temperature difference is taken, whereby the thermal conductivity can be determined.

Next, physical property values for heat capacity, thermal conductivity, and specific enthalpy are obtained (the physical property change data generation step S3).

Using these physical property values, a physical change of the substance is predicted (the physical change prediction step S5). The list of heat capacity, thermal conductivity, and specific enthalpy, and functionalized physical change data obtained up to the physical property change data generation step S3 are output, for example, to multiphysics simulation software so that physical property values required for calculation can be referenced. In this case, the physical property values may be directly written in the software, or may be read as needed when performing calculations required by the software. As prediction of a physical change of the substance, in the present case, since physical properties are mainly predicted, thermal fluid calculation may be exemplified.

In a thermal fluid calculation process using the substance as a working fluid, by using the kinematic viscosity, density, latent heat, (specific) enthalpy, thermal conductivity, isochoric heat capacity, isobaric heat capacity, diffusion coefficient, vapor pressure, excess Gibbs free energy, solvation Gibbs free energy, and Gibbs free energy calculated up to the physical property change data generation step S3, time changes of temperature distribution, pressure distribution, flow velocity distribution, and concentration distribution of the substance can be obtained. Further, time changes of temperature distribution, pressure distribution, flow velocity distribution, and concentration distribution of the substance can be obtained from the physical change data.

Finally, the predicted physical change of the substance is output (the physical change output step S6). As one example, in a flow path having an arbitrary geometric shape, time changes of temperature distribution, flow velocity distribution, and inlet/outlet temperature can be visualized.

In this manner, by using the information processing method of the above embodiment, it is possible to provide guidelines for design of equipment, required materials, or composition design thereof through temperature, flow velocity, pressure distribution, and the like. The information processing method of the above embodiment can be applied, for example, to elucidation of behavior of an automobile engine, verification of required physical properties, elucidation of a thermal cycle including piping, a compressor, a heat exchanger, and the like of an air conditioner or an air purifier, or verification of required physical properties. Further, by using the information processing method of the above embodiment, physical property values required for multiphysics simulation or mechatronics system simulation can be provided, and since the physical property values can be linked to material characteristics without experiments and from first principles, highly accurate simulation or numerical analysis can be performed.

### 1.3 Examples

Calculations were performed and comparisons were made in order to confirm the effects of the information processing method according to the present embodiment.

### 1.3.1 Example 1

In Example 1, R1234yf, which is a well-known refrigerant, was used as the substance. R1234yf is 2,3,3,3-tetrafluoropropene and is a pure substance. The chemical formula of R1234yf is C₃H₂F₄. The composition ratio of R1234yf is 100% C₃H₂F₄. Using the quantum chemical calculation software GAMESS, a most stable structure of the molecule was calculated by a Coupled Cluster method, which is an ab initio molecular orbital method, and a correlation-consistent basis set to which polarization functions were added as basis functions, thereby determining bond lengths, bond angles, and dihedral angles. Further, force constants for each of the bond lengths, bond angles, and dihedral angles were determined using the above equations (1), (2), and (3). Table 1 below shows force constants for bond lengths [kJ/(mol·nm²)] for a double bond between carbon atoms (C═C), a single bond between a carbon atom and a fluorine atom (C—F), and a single bond between carbon atoms (C—C), obtained in this manner.

TABLE 1

| Force constant for | | | Comparative |
|---|---|---|---|
| bond length | Example 1 | Reference Example | Example 1 |
| C═C | 1.2110E+05 | 1.2331E+05 | 2.8711E+05 |
| C—F | 9.6312E+04 | 9.7012E+04 | 2.9865E+05 |
| C—C | 7.8621E+04 | 8.0398E+04 | 2.7347E+05 |

In Table 1, Reference Example indicates experimental values. The experimental values of Reference Example were obtained from the literature (Zeitschrift fuer Naturforschung B (ZNB), (Germany), 2014, Vol. 69b, p. 379-387).

From comparison between Example 1 and Reference Example in Table 1, it was confirmed that calculated molecular feature values obtained by the information processing method according to the present embodiment are in good agreement with the experimental values. In particular, the calculated molecular feature values according to the information processing method of the present embodiment were within an error of 5% relative to the experimental values.

In Table 1, in Comparative Example 1, for R1234yf, a most stable molecular structure was calculated using the HF method approximated by a single Slater determinant with the quantum chemical calculation software GAMESS, and bond lengths, bond angles, and dihedral angles were determined. Charges were assigned using AM1-BCC. Force constants for bond lengths, bond angles, and dihedral angles, and the Lennard-Jones potential were defined by the Amber force field. From comparison between Comparative Example 1 and Reference Example, it can be seen that the calculated values greatly differ from the experimental values. This is considered to be due to the fact that, in the Amber force field, molecular force field parameters are defined for specific types of molecules and are not intrinsic to the molecule in question. In other words, in Example 1, which is the information processing method according to the present embodiment, a force field intrinsic to the molecule is obtained from the chemical formula of the molecule, whereas in Comparative Example 1, a default molecular force field preset for a type of molecule, rather than one intrinsic to the molecule itself, is used, and therefore the calculated values are considered to differ greatly from the experimental values.

Further, in Example 1, RESP charges and a Lennard-Jones potential were determined as the intermolecular force field. The Lennard-Jones potential of the molecule was determined so as to satisfy a relationship between distance and energy values by performing energy calculations multiple times while changing distances and angles between two different molecules.

Using the molecular feature values calculated from first principles by quantum chemical calculations in this manner, physical property values were calculated using the molecular dynamics simulation software Gromacs. For the bulk structure of the molecule, the number of molecules was set to 400, structures were randomly arranged using Pymol, followed by stable structure calculation, 100 ps annealing by NVT simulation, and then an NPT simulation at a temperature of 273 K and a pressure of 3 MPa. Density [kg/m³], specific enthalpy [kJ/kg], and specific heat at constant pressure [kJ/(kg·K)] obtained by this calculation are shown in Table 2 below.

TABLE 2

| | | Reference | Comparative |
|---|---|---|---|
| | Example 1 | Example | Example 1 |
| Density | 1168.9 | 1188.9 | 790.2 |
| Specific enthalpy | 202.15 | 200.46 | 305.6 |
| Specific heat at | 1.2689 | 1.2718 | 1.1013 |
| constant pressure | | | |

In Table 2, Reference Example indicates values obtained from REFPROP (Reference Fluid Thermodynamic and Transport Properties Database) provided by NIST (National Institute of Standards and Technology). Since REFPROP is a database compiling experimental results, values obtained from REFPROP are adopted as the Reference Example indicating experimental values.

From comparison between Example 1 and Reference Example in Table 2, it was confirmed that the calculated physical property values obtained by the information processing method according to the present embodiment are in good agreement with the experimental values.

In Table 2, Comparative Example 1 indicates density [kg/m³], specific enthalpy [kJ/kg], and specific heat at constant pressure [kJ/(kg·K)] obtained by the same method as in Example 1 using molecular feature values obtained using the Amber force field as described above. From comparison between Reference Example and Comparative Example 1, it can be seen that the calculated values greatly differ from the experimental values. This is considered to be because the calculated molecular feature values in Comparative Example 1 greatly differ from the experimental values.

Subsequently, in Example 1, using the bulk structure obtained based on the NPT simulation, an NVE simulation was performed for 3 ns at the same volume, and a kinematic viscosity and thermal conductivity were calculated from an autocorrelation function obtained in the last 1 ns. Further, a diffusion coefficient was calculated using Gromacs from an autocorrelation function using a bulk structure obtained by an NPT simulation at a temperature of 303.2 K and a pressure of 1 atm. The kinematic viscosity [cm²/s], thermal conductivity [mW/(m·K)], and diffusion coefficient [m²/s] obtained by these calculations are shown in Table 3 below.

TABLE 3

| | | Reference | Comparative | Comparative |
|---|---|---|---|---|
| | Example 1 | Example | Example 1 | Example 2 |
| Kinematic | 0.0017435 | 0.0017991 | 0.0013224 | N.D. |
| viscosity | | | | |
| Thermal | 72.956 | 73.235 | 65.438 | N.D. |
| conductivity | | | | |
| Diffusion | 0.111 | 0.108 | — | N.D. |
| coefficient | | | | |

In Table 3, the kinematic viscosity and thermal conductivity of Reference Example indicate values obtained from REFPROP. The experimental value of the thermal diffusion coefficient of Reference Example was obtained from the literature (Netsu Bussei, 2020, Vol. 34, Issue 1, p. 10-14).

From comparison between Example 1 and Reference Example in Table 3, it was confirmed that the calculated physical property values obtained by the information processing method according to the present embodiment are in good agreement with the experimental values. In particular, the calculated physical property values according to the information processing method of the present embodiment were within an error of approximately 10% relative to the experimental values.

In Table 3, Comparative Example 1 indicates the kinematic viscosity and thermal conductivity obtained by the same method as in Example 1 using molecular feature values obtained using the Amber force field as described above. From comparison between Reference Example and Comparative Example 1, it can be seen that the calculated values greatly differ from the experimental values. This is considered to be because the calculated molecular feature values in Comparative Example 1 greatly differ from the experimental values.

In Table 3, Comparative Example 2 corresponds to the GEMC simulation described in Non-Patent Document 1; however, since the kinematic viscosity, thermal conductivity, and diffusion coefficient are, in principle, impossible to calculate, they are indicated as “N.D.”

Further, in Example 1, vapor pressure was determined. Specifically, an NPT simulation was performed at a temperature of 303 K and a pressure of 0.1 MPa, and then, while fixing the temperature, a pressure increment interval at which density changes by 100 kg/m³ or more was determined. For the obtained pressure increment interval, pressure was varied more finely to perform calculations, and a pressure at which density changes by 100 kg/m³ or more was determined as the vapor pressure. Table 4 shows the vapor pressure [MPa] obtained by this calculation, latent heat [kJ/kg] obtained by an NVT simulation while changing temperature so that the vapor pressure becomes 1 atm, and liquid density [kg/m³] and gas density [kg/m³] at that time.

TABLE 4

| | | | Comparative |
|---|---|---|---|
| | Example 1 | Reference Example | Example 2 |
| vapor pressure | 0.7775 | 0.78033 | 0.735 |
| Latent heat | 175.49 | 179.97 | 131.1 |
| Liquid density | 1070.5 | 1073.9 | 1075.1 |
| Gas density | 43.31 | 43.544 | 43.16 |

In Table 4, Reference Example indicates values obtained from REFPROP.

From comparison between Example 1 and Reference Example in Table 4, it was confirmed that the calculated physical property values obtained by the information processing method according to the present embodiment are in good agreement with the experimental values.

In Table 4, Comparative Example 2 indicates values obtained by the GEMC simulation described in Non-Patent Document 1. From comparison between Reference Example and Comparative Example 2, it can be seen that the calculated values greatly differ from the experimental values. This is considered to be because the calculated molecular feature values in Comparative Example 2 greatly differ from the experimental values. Further, in Example 1, excess Gibbs free energy and Gibbs free energy were determined. Specifically, at a temperature of 243 K and a pressure of 2 MPa, NPT simulations of 500 molecules and 501 molecules were performed for 2 ns, and an additional 10 ps calculation was performed as a main calculation. Further, for one molecule in a cell having the same volume as that of the 500 molecules, an NVT simulation was performed for 100 ps as a main calculation. Using the three types of calculation systems, excess Gibbs free energy was calculated using an ER calculation. Table 5 shows the excess Gibbs free energy [kJ/kg] and the Gibbs free energy [kJ/kg] at that time.

TABLE 5

| | | Reference | Comparative |
|---|---|---|---|
| | Example 1 | Example | Example 1 |
| Excess Gibbs free | −95.91 | −95.535 | −69.823 |
| energy | | | |
| Gibbs free energy | −44.213 | −43.838 | −25.61 |

In Table 5, the excess Gibbs free energy and Gibbs free energy of Reference Example indicate values obtained from REFPROP. From comparison between Reference Example and Comparative Example 1, it can be seen that the calculated values greatly differ from the experimental values. This is considered to be because the calculated molecular feature values in Comparative Example 1 greatly differ from the experimental values. In comparison therewith, the excess Gibbs free energy and Gibbs free energy of Example 1 are considerably close to those of Reference Example 1.

A thermal fluid calculation in piping was performed for Example 1 obtained as described above. The substance of Example 1 at 200 K was caused to flow from the left at a flow velocity of 1.0 m/s in a pipe having a radius of 0.5 m, and a region from 1.5 m to 2.5 m from the left end was heated to 400 K so that latent heat was generated at a boiling point (dew point) at the vapor pressure obtained in Table 4, thereby causing vaporization. At that time, the liquid density and gas density obtained in Table 4 were used as densities. Further, the kinematic viscosity, thermal conductivity, and diffusion coefficient were determined in the same manner as in Table 3, and the specific heat at constant pressure and specific enthalpy were determined for required temperatures and pressures in the same manner as in Table 2.

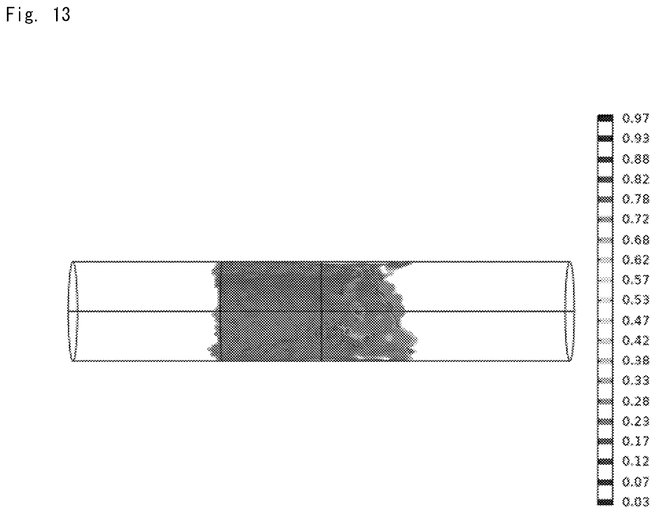

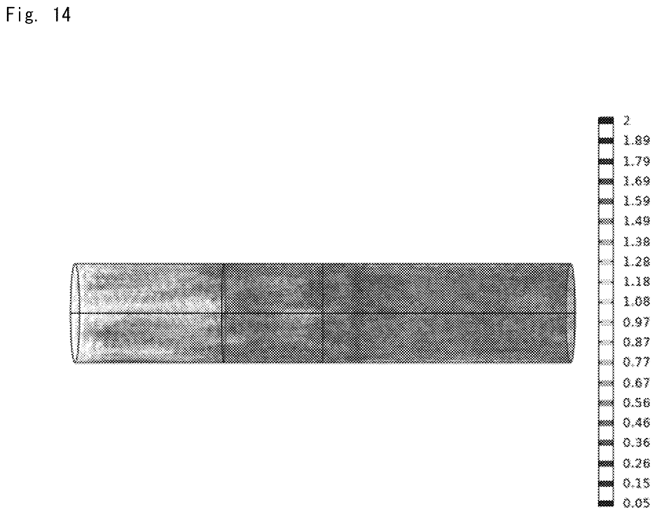

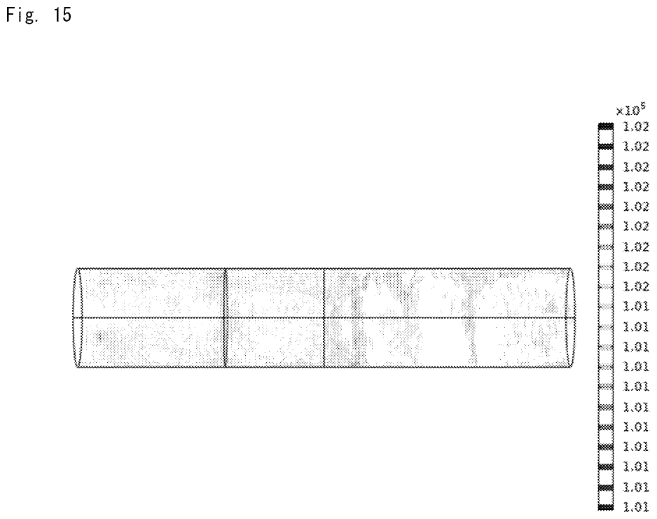

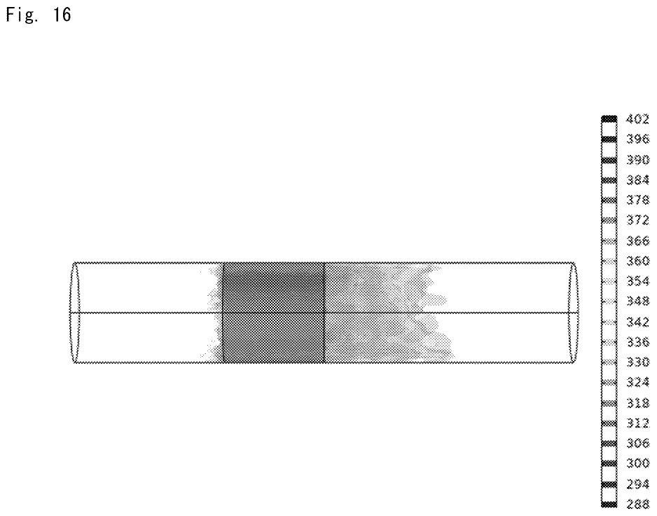

Next, results of thermal fluid calculation for a gas-liquid two-phase flow are shown. FIG. 13 is an illustrative diagram of a gas distribution in the thermal fluid calculation of Example 1. FIG. 14 is an illustrative diagram of a flow velocity distribution in the thermal fluid calculation of Example 1. FIG. 15 is an illustrative diagram of a pressure distribution in the thermal fluid calculation of Example 1. FIG. 16 is an illustrative diagram of a temperature distribution in the thermal fluid calculation of Example 1. FIG. 13 to FIG. 16 each show distributions 5 seconds after start of the calculation. By using the physical property values according to the information processing method of the present embodiment, physical property values can be predicted without experiments, and physical changes such as those obtained from thermal fluid calculations can be predicted.

### 1.3.2 Example 2

In Example 2, R454B was used as the substance. Since R454B contains a plurality of types of molecules, it is a mixture. More specifically, R454B is a mixed refrigerant including R1234yf and R32. R32 is difluoromethane, and its chemical formula is CH₂F₂. Molecular feature values were determined in the same manner as for R1234yf in Example 1.

In R454B, a composition ratio of R1234yf to R32 was set to 68.1:31.9 by mass ratio. The number of molecules of R454B was defined based on this composition ratio, structures were randomly arranged using Pymol, followed by stable structure calculation, 100 ps annealing by NVT simulation, and then an NPT simulation at a temperature of 273 K and a pressure of 3 MPa. Density [kg/m³], specific enthalpy [kJ/kg], and specific heat at constant pressure [kJ/(kg·K)] obtained by this calculation are shown in Table 6 below.

TABLE 6

| | Example 2 | Reference Example |
|---|---|---|
| Density | 1101.4 | 1091.5 |
| Specific enthalpy | 201.93 | 199.98 |
| Specific heat at | 1.5761 | 1.5967 |
| constant pressure | | |

In Table 6, Reference Example indicates values obtained from REFPROP.

From comparison between Example 2 and Reference Example in Table 6, it was confirmed that the calculated physical property values obtained by the information processing method according to the present embodiment are in good agreement with the experimental values.

Further, in Example 2, solvation Gibbs free energy and Gibbs free energy were determined. Specifically, at a temperature of 243 K and a pressure of 2 MPa, NPT simulations of 500 molecules and 501 molecules obtained by adding one molecule of R1234yf were performed for 2 ns, and an additional 10 ps calculation was performed as a main calculation. Further, for one molecule in a cell having the same volume as that of the 500 molecules, an NVT simulation was performed for 100 ps as a main calculation. Using the three types of calculation systems, excess Gibbs free energy was calculated using an ER calculation. Table 7 shows the solvation Gibbs free energy [kJ/kg] and the Gibbs free energy [kJ/kg] at that time.

TABLE 7

| | Example 2 | Reference Example |
|---|---|---|
| Solvation Gibbs free | −108.75 | −110.73 |
| energy | | |
| Gibbs free energy | −42.102 | −44.678 |

In Table 7, the solvation Gibbs free energy and Gibbs free energy of Reference Example indicate values obtained from REFPROP. The solvation Gibbs free energy and Gibbs free energy of R454B in Example 2 are judged to be considerably close to those of Reference Example 1.

### 1.4 Advantageous Effects

The above-described information processing method is an information processing method executed by the computer system 1, the method comprising: acquiring one or more chemical formulas of respective one or more types of molecules constituting a substance and a composition ratio of the one or more types of molecules (S1); calculating, from the one or more chemical formulas of the respective one or more types of molecules, one or more molecular feature values of the respective one or more types of molecules (S2); and generating physical property change data representing a change in a physical property value of the substance by using the composition ratio and the one or more molecular feature values (S3). This configuration enables calculation of a physical property value of the substance without experimental values regarding the molecules constituting the substance. Furthermore, this configuration enables output of the calculated physical property value.

The information processing method further includes executing a simulation using the physical property change data as input based on setting of a geometric shape condition (S4) to predict a physical change of the substance (S5). This configuration enables prediction of a physical change of the substance.

The information processing method further includes issuing an instruction to execute a simulation using the physical property change data as input (S5) and outputting prediction data of a physical change of the substance calculated in the simulation (S6). This configuration enables prediction and output of a physical change of the substance.

In the information processing method, calculating the one or more molecular feature values includes performing a quantum chemical calculation on the one or more chemical formula of the respective one or more types of molecules to determine the one or more molecular feature values from first principles. This configuration enables acquisition of a change in a physical property value of the substance solely by calculation without experimental values regarding the molecules constituting the substance.

In the information processing method, calculating the one or more molecular feature values is determined by obtaining a most stable point between two or more molecules through a stable structure calculation with a fixed structure in a quantum chemical calculation, with regard to van der Waals potential parameters in the one or more molecular feature values. This configuration enables acquisition of a change in a physical property value of the substance solely by calculation without experimental values regarding the molecules constituting the substance.

In the information processing method, generating the physical property change data includes calculating, by a molecular dynamics simulation using the composition ratio and the one or more molecular feature values, a physical structure representing a state formed by the substance. This configuration enables acquisition of a change in a physical property value of the substance solely by calculation without experimental values regarding the molecules constituting the substance.

In the information processing method, the molecular dynamics simulation performs at least one of an NVT simulation, an NPT simulation, an NPE simulation, a BAR calculation, or an ER calculation. This configuration enables acquisition of a change in a physical property value of the substance solely by calculation without experimental values regarding the molecules constituting the substance.

In the information processing method, generating the physical property change data further includes calculating the physical property value by a statistical mechanics calculation based on the physical structure. This configuration enables acquisition of a change in a physical property value of the substance solely by calculation without experimental values regarding the molecules constituting the substance.

In the information processing method, the statistical mechanics calculation is determined by an ensemble average and/or an autocorrelation function based on the Green-Kubo formula. This configuration enables acquisition of a change in a physical property value of the substance solely by calculation without experimental values regarding the molecules constituting the substance.

In the information processing method, generating the physical property change data includes calculating, by a molecular dynamics simulation, a physical structure for each of one target molecule, N molecules other than the target molecule, and a total of N+1 molecules including the target molecule and the molecules other than the target molecule, and calculating an energy relaxed when one molecule is added to the N molecules by subtracting, from an energy calculated from the physical structure of the N+1 molecules, an energy calculated when one target molecule is added to the N molecules. This configuration enables calculation of excess Gibbs free energy.

In the information processing method, the physical property value includes at least one of a kinematic viscosity, density, latent heat, (specific) enthalpy, thermal conductivity, isochoric heat capacity, isobaric heat capacity, diffusion coefficient, vapor pressure, excess Gibbs free energy, solvation Gibbs free energy, and Gibbs free energy. This configuration enables calculation of a physical property value of the substance without experimental values regarding the molecules constituting the substance.

In the information processing method, the physical change data representing a change in the physical property value includes a continuous function with respect to the physical property value, generated based on a plurality of discrete values of a physical state of the substance. This configuration enables output of physical property values corresponding to various inputs.

The above-described presentation method is executed by the computer system 1, and includes receiving input of one or more chemical formulas of respective one or more molecules constituting a substance and a composition ratio of the one or more molecules, generating an image G1, G2 indicating continuous data for each physical property value required for thermal fluid calculation based on the one or more chemical formulas and the composition ratio which are input, and presenting the image G1, G2 generated. This configuration enables easy understanding of changes in physical property values of the substance. Further, this configuration enables the obtained continuous data for each physical property value to be transferred to and used by other programs or analysis methods.

The above information processing method can be realized by the computer system 1 executing a program. This program is a program for causing the computer system 1 to execute the above information processing method. This configuration enables calculation of a physical property value of the substance without experimental values regarding the molecules constituting the substance.

The above-described manufacturing method is a method for manufacturing physical property change data of a substance, executed by the computer system 1, the method comprising acquiring one or more chemical formulas of respective one or more molecules constituting the substance and a composition ratio of the one or more molecules, determining, from the one or more chemical formulas of the respective one or more molecules, one or more molecular feature values of the respective one or more molecules, and generating physical property change data representing a change in a physical property value of the substance by using the composition ratio and the one or more molecular feature values. This configuration enables calculation of a physical property value of the substance without experimental values regarding the molecules constituting the substance.

### 2. Variations

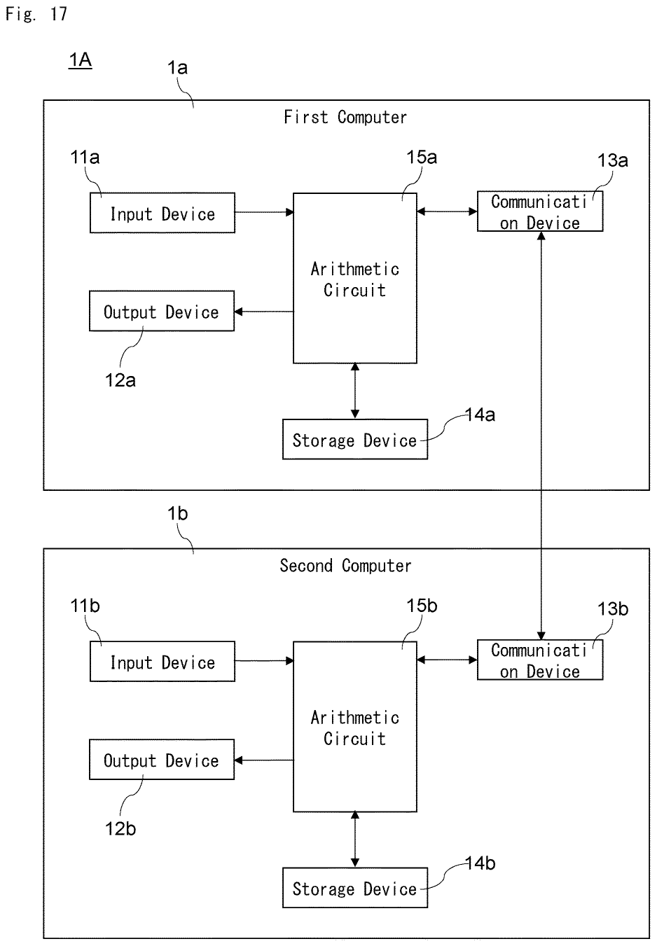

FIG. 17 is a block diagram of a computer system 1A according to a variation. The computer system 1A may be implemented by a plurality of computers including a first computer 1a and a second computer 1b.

Each of the first computer 1a and the second computer 1b may be implemented by a server, a personal computer (desktop computer, laptop computer), a mobile terminal (smartphone, tablet terminal, etc.), or the like.

The first and second computers 1a and 1b include input devices 11a and 11b, output devices 12a and 12b, communication devices 13a and 13b, storage devices 14a and 14b, and arithmetic circuit 15a and 15b, respectively. The input devices 11a and 11b, the output devices 12a and 12b, the communication devices 13a and 13b, the storage devices 14a and 14b, and the arithmetic circuits 15a and 15b are respectively the same as the input device 11, the output device 12, the communication device 13, the storage device 14, and the arithmetic circuit 15.

In this variation, the arithmetic circuits 15a and 15b share execution of the acquisition step S1, the molecular feature value determination step S2, the physical property change data generation step S3, the geometric shape condition setting step S4, the physical change prediction step S5, and the physical change output step S6.

As one example, the arithmetic circuit 15a executes the acquisition step S1, the molecular feature value determination step S2, and the physical property change data generation step S3, and the arithmetic circuit 15b executes the geometric shape condition setting step S4, the physical change prediction step S5, and the physical change output step S6.

In this case, the first computer 1a executes the acquisition step S1, the molecular feature value determination step S2, and the physical property change data generation step S3 by the arithmetic circuit 15a to obtain physical property change data. The first computer 1a transmits the obtained physical property change data to the second computer 1b via the communication device 13a. The second computer 1b receives the physical property change data from the first computer 1a via the communication device 13b. In the second computer 1b, the arithmetic circuit 15b executes the geometric shape condition setting step S4, the physical change prediction step S5, and the physical change output step S6 using the physical property change data.

In this example, the first computer 1a is responsible for calculation of physical property values, and the second computer 1b is responsible for simulation using the physical property values. The second computer 1b may be replaced by a computer that executes conventionally known simulation software.

As is clear from the above embodiment and variation, the computer system 1 may be implemented by a single computer or may be implemented by a plurality of computers (capable of communicating with each other). It is not essential that a plurality of functions (components) in the computer system 1 be integrated within a single housing, and components of the computer system 1 may be distributed among a plurality of housings. Furthermore, at least part of the functions of the computer system 1, for example, part of the functions of the arithmetic circuit 15, may be implemented by a cloud (cloud computing) or the like.

The computer system 1A does not necessarily have to include the second computer 1b.

### 3. Aspects

As is clear from the above embodiment and variations, the present disclosure includes the following aspects.

### Aspect 1

An information processing method executed by a computer system, including:
acquiring one or more chemical formulas of respective one or more types of molecules constituting a substance and a composition ratio of the one or more types of molecules;
calculating, from the one or more chemical formulas of the respective one or more types of molecules, one or more molecular feature values of the respective one or more types of molecules; and
generating physical property change data representing a change in a physical property value of the substance by using the composition ratio and the one or more molecular feature values.

### Aspect 2

The information processing method of aspect 1, further comprising executing a simulation using the physical property change data as input based on setting of a geometric shape condition to predict a physical change of the substance.

### Aspect 3

The information processing method of aspect 1 or 2, further comprising issuing an instruction to execute a simulation using the physical property change data as input, and outputting prediction data of a physical change of the substance calculated in the simulation.

### Aspect 4

The information processing method of any one of aspects 1 to 3, wherein calculating the one or more molecular feature values includes performing a quantum chemical calculation on the one or more chemical formulas of the respective one or more types of molecules to determine the one or more molecular feature values from first principles.

### Aspect 5

The information processing method of any one of aspects 1 to 4, wherein calculating the one or more molecular feature values is determined by obtaining a most stable point between two or more molecules through a stable structure calculation with a fixed structure in a quantum chemical calculation, with regard to one or more van der Waals potential parameters in the one or more molecular feature values.

### Aspect 6

The information processing method of any one of aspects 1 to 5, wherein generating the physical property change data includes calculating, by a molecular dynamics simulation using the composition ratio and the one or more molecular feature values, a physical structure representing a state formed by the substance.

### Aspect 7

The information processing method of aspect 6, wherein the molecular dynamics simulation includes performing at least one of an NVT simulation, an NPT simulation, an NPE simulation, a BAR calculation, or an ER calculation.

### Aspect 8

The information processing method of aspect 6 or 7, wherein generating the physical property change data further includes calculating the physical property value by a statistical mechanics calculation based on the physical structure.

### Aspect 9

The information processing method of aspect 8, wherein the statistical mechanics calculation is determined by an ensemble average and/or an autocorrelation function based on the Green-Kubo formula.

### Aspect 10

The information processing method of any one of aspects 1 to 9, wherein generating the physical property change data includes:
calculating, by a molecular dynamics simulation, a physical structure for each of one target molecule, N molecules other than the target molecule, and a total of N+1 molecules including the target molecule and the molecules other than the target molecule; and
calculating an energy relaxed when one molecule is added to the N molecules by subtracting, from an energy calculated from the physical structures of the N+1 molecules, an energy calculated when one target molecule is added to the N molecules.

### Aspect 11

The information processing method of any one of aspects 1 to 10, wherein the physical property value includes at least one of a kinematic viscosity, density, latent heat, (specific) enthalpy, thermal conductivity, isochoric heat capacity, isobaric heat capacity, diffusion coefficient, vapor pressure, excess Gibbs free energy, solvation Gibbs free energy, and Gibbs free energy.

### Aspect 12

The information processing method of any one of aspects 1 to 11, wherein the physical property change data representing a change in the physical property value includes a continuous function with respect to the physical property value, generated based on a plurality of discrete values of a physical state of the substance.

### Aspect 13

A presentation method for thermal fluid analysis, executed by a computer system, comprising:
receiving input of one or more chemical formulas of respective one or more molecules constituting a substance and a composition ratio of the one or more molecules;
generating an image indicating continuous data for each physical property value required for thermal fluid analysis based on the one or more chemical formulas and the composition ratio which are input; and
presenting the image generated.

### Aspect 14

A program for causing a computer system to execute the information processing method of any one of aspects 1 to 12.

### Aspect 15

A manufacturing method for physical property change data of a substance, executed by a computer system, including:
acquiring one or more chemical formulas of respective one or more molecules constituting a substance and a composition ratio of the one or more molecules;
determining, from the one or more chemical formulas of the respective one or more molecules, one or more molecular feature values of the respective one or more molecules; and
generating physical property change data representing a change in a physical property value of the substance by using the composition ratio and the one or more molecular feature values.

### Aspect 21

A prediction method for a physical change of a substance, executed by a computer system, including:
acquiring one or more chemical formulas of respective one or more molecules constituting the substance and a composition ratio of the one or more molecules;
determining, from the one or more chemical formulas of the respective one or more molecules, one or more molecular feature values of the respective one or more molecules;
determining, from the composition ratio and the one or more molecular feature values, a physical state of the substance including at least one of a structure of the substance or a thermal physical property value of the substance;
generating physical change data representing a change in the physical state based on the physical state; and
predicting the physical change using the physical change data.

### Aspect 22

The prediction method of aspect 21, wherein the one or more molecular feature values are determined from chemical formulas of a plurality of different molecules by quantum chemical calculation from first principles.

### Aspect 23

The prediction method of aspect 21 or 22, wherein the one or more molecular feature values include a molecular structure and a molecular force field.

### Aspect 24

The prediction method of aspect 23, wherein:
the molecular structure is determined based on a most stable structure obtained by quantum chemical calculation from the one or more chemical formulas of the respective molecules; and
the molecular force field is determined based on the molecular structure.

### Aspect 25

The prediction method of aspect 24, wherein:
the molecular structure includes at least one of a bond length, a bond angle, or a dihedral angle;
the molecular force field includes an intramolecular force field; and
the intramolecular force field includes a force constant for at least one of the bond length, the bond angle, or the dihedral angle.

### Aspect 26

The prediction method of aspect 25, wherein an error of a force constant of the intramolecular force field relative to a force constant calculated from an actually measured value is within 5%.

### Aspect 27

The prediction method of any one of aspects 24 to 26, wherein:
the molecular force field includes an intermolecular force field; and
the intermolecular force field includes at least one of a molecular charge or a van der Waals potential.

### Aspect 28

The prediction method of any one of aspects 21 to 27, wherein the thermal physical property value includes at least one of a kinematic viscosity, density, latent heat, (specific) enthalpy, thermal conductivity, isochoric heat capacity, isobaric heat capacity, diffusion coefficient, vapor pressure, or entropy.

### Aspect 29

The prediction method of any one of aspects 21 to 28, wherein the physical change data includes a continuous function of the physical state generated based on a plurality of discrete values of the physical state of the substance.

### Aspect 30

The prediction method of any one of aspects 21 to 29, wherein predicting the physical change includes at least one of:
calculating a change in the physical state used for simulation of a thermal fluid cycle in a geometric shape, computational fluid dynamics, or thermal fluid analysis;
calculating a change in the physical state for design optimization of simulation of a thermal fluid cycle in a geometric shape or computational fluid dynamics; or
calculating a change in the physical state to generate thermal physical property data used for simulation of a thermal fluid cycle in a geometric shape or computational fluid dynamics.

### Aspect 31

A program for causing the computer system to execute the prediction method of any one of aspects 21 to 30.

### Aspect 32

A manufacturing method for physical change data of a mixture composed of a plurality of different molecules, executed by a computer system, including:
acquiring chemical formulas of the plurality of different molecules and a mixing ratio of the plurality of molecules;
determining molecular feature values of the plurality of different molecules from the chemical formulas of the plurality of different molecules;
determining, from the molecular feature values and the mixing ratio, a physical state of the mixture including at least one of a structure of the mixture and a thermal physical property value; and
generating physical change data representing a change in the physical state based on the physical state.

### Aspect 33

An information processing method executed by a computer system, including:
acquiring one or more chemical formulas of respective one or more molecules constituting a substance and a composition ratio of the one or more molecules;
determining, based on the one or more chemical formulas and the composition ratio, a physical state of the substance including at least one of a structure of the substance and a thermal physical property value of the substance; and
outputting a physical change of the substance derived based on the physical state, the physical change including at least one of a change in the structure or a change in the thermal physical property value.

### Aspect 34

The information processing method of aspect 33, wherein outputting the physical change includes generating an image indicating at least one of different states of the structure or different values of the thermal physical property value, and outputting the generated image.

### Aspect 35

A program for causing a computer system to execute the information processing method of aspect 33 or 34.

Aspects 22 to 29 are optional and not essential. Aspects 22 to 29 can be combined with Aspects 32, 33, and 34 appropriately.

### INDUSTRIAL APPLICABILITY

The present disclosure is applicable to an information processing method, a presentation method, a program, and a manufacturing method. Specifically, the present disclosure is applicable to an information processing method for calculating a change in a physical property value of a substance, a presentation method, a program for causing a computer system to execute the information processing method, and a manufacturing method for manufacturing physical property change data.

### REFERENCE SIGNS LIST

S1 Acquisition Step
S2 Molecule Feature Value Determination Step
S3 Physical Property Change Data Generation Step

1. An information processing method executed by a computer system, comprising:
acquiring one or more chemical formulas of respective one or more types of molecules constituting a substance and a composition ratio of the one or more types of molecules;
calculating, from the one or more chemical formulas of the respective one or more types of molecules, one or more molecular feature values of the respective one or more types of molecules; and
generating physical property change data representing a change in a physical property value of the substance by using the composition ratio and the one or more molecular feature values.

2. The information processing method of claim 1, further comprising executing a simulation using the physical property change data as input based on setting of a geometric shape condition to predict a physical change of the substance.

3. The information processing method of claim 1, further comprising issuing an instruction to execute a simulation using the physical property change data as input; and
outputting prediction data of a physical change of the substance calculated in the simulation.

4. The information processing method of claim 1, wherein calculating the one or more molecular feature values includes performing a quantum chemical calculation on the one or more chemical formulas of the respective one or more types of molecules to determine the one or more molecular feature values from first principles.

5. The information processing method of claim 1, wherein calculating the one or more molecular feature values is determined by obtaining a most stable point between two or more molecules through a stable structure calculation with a fixed structure in a quantum chemical calculation, with regard to one or more van der Waals potential parameters in the one or more molecular feature values.

6. The information processing method of claim 1, wherein generating the physical property change data includes calculating, by a molecular dynamics simulation using the composition ratio and the one or more molecular feature values, a physical structure representing a state formed by the substance.

7. The information processing method of claim 6, wherein the molecular dynamics simulation includes performing at least one of an NVT simulation, an NPT simulation, an NPE simulation, a BAR calculation, or an ER calculation.

8. The information processing method of claim 6, wherein generating the physical property change data further includes calculating the physical property value by a statistical mechanics calculation based on the physical structure.

9. The information processing method of claim 8, wherein the statistical mechanics calculation is determined by an ensemble average and/or an autocorrelation function based on the Green-Kubo formula.

10. The information processing method of claim 1, wherein generating the physical property change data includes:
calculating, by a molecular dynamics simulation, a physical structure for each of one target molecule, N molecules other than the target molecule, and a total of N+1 molecules including the target molecule and the molecules other than the target molecule; and
calculating an energy relaxed when one molecule is added to the N molecules by subtracting, from an energy calculated from the physical structures of the N+1 molecules, an energy calculated when one target molecule is added to the N molecules.

11. The information processing method of claim 1, wherein the physical property value includes at least one of a kinematic viscosity, density, latent heat, (specific) enthalpy, thermal conductivity, isochoric heat capacity, isobaric heat capacity, diffusion coefficient, vapor pressure, excess Gibbs free energy, solvation Gibbs free energy, and Gibbs free energy.

12. The information processing method of claim 1, wherein the physical property change data representing a change in the physical property value includes a continuous function with respect to the physical property value, generated based on a plurality of discrete values of a physical state of the substance.

13. A presentation method for thermal fluid analysis, executed by a computer system, comprising:
receiving input of one or more chemical formulas of respective one or more molecules constituting a substance and a composition ratio of the one or more molecules;
generating an image indicating continuous data for each physical property value required for thermal fluid analysis based on the one or more chemical formulas and the composition ratio which are input; and
presenting the image generated.

14. A non-transitory computer readable medium storing a program for causing a computer system to execute the information processing method of claim 1.

15. A manufacturing method for physical property change data of a substance, executed by a computer system, comprising:
acquiring one or more chemical formulas of respective one or more molecules constituting a substance and a composition ratio of the one or more molecules;
determining, from the one or more chemical formulas of the respective one or more molecules, one or more molecular feature values of the respective one or more molecules; and
generating physical property change data representing a change in a physical property value of the substance by using the composition ratio and the one or more molecular feature values.
